(19) United States
(12) Patent Application Publication
(10) Pub. No.: US 2007/0142411 A1
(43) Pub. Date: Jun. 21, 2007

(54) Use of cyclooxygenase-2 selective inhibitors for the treatment of schizophrenic disorders

(76) Inventors: James Hagan, Harlow (GB); Carol Routledge, Harlow (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/595,864

(22) PCT Filed: Nov. 17, 2004
(86) PCT No.: PCT/EP04/13076

§ 371 Date: Feb. 21, 2007

(30) Foreign Application Priority Data
Nov. 19, 2003 (GB) 0326967.7
Dec. 2, 2003 (GB) 0327937.9

(51) Int. Cl. A61K 31/513

(52) U.S. Cl. 514/269

(57) ABSTRACT

The invention concerns the use of compounds of formula (I), (II) and (III) 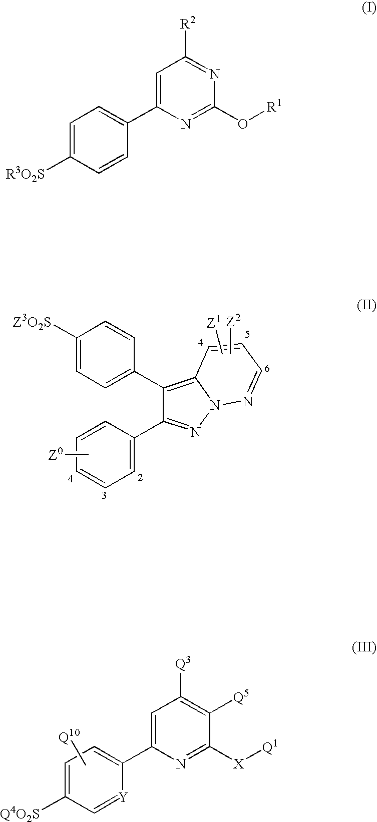 which are COX-2 (cyclooxygenase-2) inhibitors, and pharmaceutically acceptable salts and solates thereof, for the treatment of schizophrenic disorders. Schizophrenic disorders of the invention are to be intended schizophrenia, delusional disorders, affective disorders, autism or tic disorders, schizophreniform disorders, in particular chronic schizophrenic psychoses and schizoaffective psychoses, temporary acute psychotic disorders. Moreover, the invention is concerned with the use of a pyrimidine derivative known as COX-2 inhibitor in combination with a neuroleptic drug for the treatment of schizophrenic disorders such as those defined above.

The invention concerns the use of compounds, which are selective COX-2 (cyclooxygenase-2) inhibitors, for the treatment of schizophrenic disorders. Schizophrenic disorders of the invention include schizophrenia, delusional disorders, affective disorders, autism or tic disorders, schizophreniform disorders, in particular chronic schizophrenic psychoses and schizoaffective psychoses and temporary acute psychotic disorders.

Moreover, the invention is concerned with the use of a compound of the present invention in combination with a neuroleptic drug for the treatment of the above mentioned schizophrenic disorders.

In one embodiment, the present invention provides a new use of compounds of formula (I) 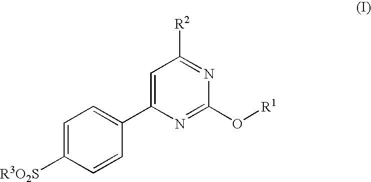
and pharmaceutically acceptable salts or solvates thereof, wherein
R¹ is selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl, A(CR⁴R⁵)ₙ and B(CR⁴R⁵)ₙ;
R² is C₁₋₂alkyl substituted by one to five fluorine atoms;
R³ is selected from the group consisting of C₁₋₆alkyl, NH₂ and R⁷CONH;
R⁴ and R⁵ are independently selected from H or C₁₋₆alkyl;
A is selected from the group consisting of unsubstituted 5- or 6-membered heteroaryl, unsubstituted 6-membered aryl, 5- or 6-membered heteroaryl substituted by one or more R⁶ and 6-membered aryl substituted by one or more R⁶;
R⁶ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B is a ring selected from the group consisting of 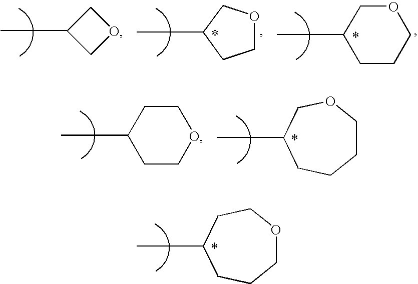
where 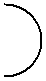
defines the point of attachment of the ring;
R⁷ is selected from the group consisting of H, C₁₋₆alkyl, C₁₋₆alkoxy, C₁₋₆-alkylOC₁₋₆alkyl, phenyl, HO₂CC₁₋₆alkyl, C₁₋₆alkylOCOC₁₋₆alkyl, C₁₋₆-alkylOCO, H₂NC₁₋₆alkyl, C₁₋₆-alkylOCONHC₁₋₆-alkyl and C₁₋₆alkylCONHC₁₋₆-alkyl; and
n is 0 to 4.

The term halogen is used to represent fluorine, chlorine, bromine or iodine.

The term ‘alkyl’ as a group or part of a group means a straight or branched chain alkyl group, for example a methyl, ethyl, n-propyl, i-propyl, n-butyl, s-butyl or t-butyl group.

The term 5-membered heteroaryl means a heteroaryl selected from the following:

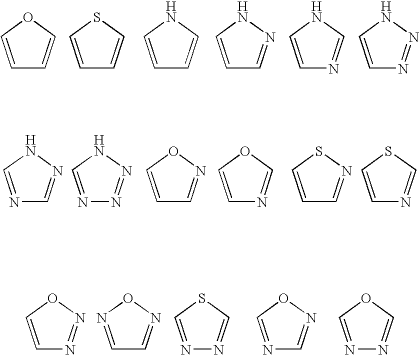

The term 6-membered heteroaryl means a heteroaryl selected from the following:

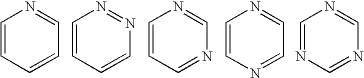

The term 6-membered aryl means:

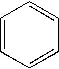

It is to be understood that the present invention encompasses all isomers of the compounds of formula (I) and their pharmaceutically acceptable derivatives, including all geometric, tautomeric and optical forms, and mixtures thereof (e.g. racemic mixtures). In particular when the ring B lacks a plane of symmetry the compounds of formula (I) contain a chiral centre as indicated therein by the asterisk *. Furthermore, it will be appreciated by those skilled in the art that when R⁴ and R⁵ in formula (I) are different the corresponding compounds contain at least one chiral centre, by virtue of the asymmetric carbon atom defined thereby, and that such compounds exist in the form of a pair of optical isomers (i.e. enantiomers).

In one aspect of the invention R¹ is selected from the group consisting of H, C₁₋₆alkyl, C₁₋₂alkyl substituted by one to five fluorine atoms, C₃₋₆alkenyl, C₃₋₆alkynyl, C₃₋₁₀cycloalkylC₀₋₆alkyl, C₄₋₁₂bridged cycloalkyl and B(CR⁴R⁵)ₙ;

In another aspect of the invention R¹ is C₁₋₆alkyl or C₁₋₂alkyl substituted by one to five fluorine atoms. In another aspect R¹ is C₂₋₆alkyl (e.g. n-butyl).

In another aspect of the invention R¹ is C₃₋₁₀cycloalkylC₀₋₆alkyl, such as C₃₋₁₀cycloalkyl (e.g. cyclopentyl or cyclohexyl). In another aspect R¹ is C₃₋₁₀cycoalkylmethyl, such as C₃₋₇cycloalkylmethyl (e.g. cyclopentylmethyl).

In another aspect of the invention R¹ is A(CR⁴R⁵)ₙ.

In another aspect of the invention R² is CHF₂, CH₂F or CF₃. In another aspect R² is CF₃.

In another aspect of the invention R³ is C₁₋₆alkyl, such as C₁₋₃alkyl (e.g. methyl).

In another aspect of the invention R⁴ and R⁵ are independently selected from H or methyl.

In another aspect R⁴ and R⁵ are both H.

In another aspect of the invention A is selected from the group consisting of 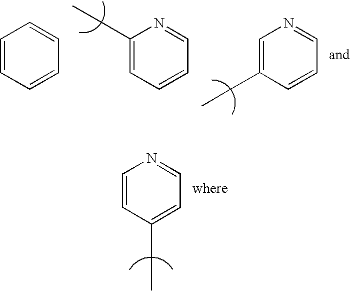 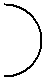
defines the point of attachment of the ring and A is unsubstituted or substituted by one or two R⁶.

In another aspect of the invention R⁶ is selected from the group consisting of halogen (e.g. F), C₁₋₃alkyl (e.g. methyl), C₁₋₃alkyl substituted by one to three fluorine atoms (e.g. CF₃), and C₁₋₃alkoxy (e.g. methoxy).

In another aspect of the invention R⁷ is selected from the group consisting of C₁₋₆alkyl (e.g. ethyl), phenyl and aminomethyl.

In another aspect of the invention n is 1 to 4.

In another aspect of the invention n is 0 to 2 (e.g. 0).

It is to be understood that the invention covers all combinations of particular aspects of the invention as described hereinabove.

Within the invention there is provided one group of compounds of formula (I) (group A) wherein: R¹ is C₁₋₆alkyl (e.g. n-butyl); R² is CF₃; and R³ is C₁₋₆alkyl, such as C₁₋₃alkyl (e.g. methyl).

Within the invention there is provided another group of compounds of formula (I) (group B) wherein: R¹ is C₃₋₁₀cycloalkylC₀₋₆alkyl, such as C₃₋₁₀cycloalkyl (e.g. cyclopentyl or cyclohexyl); R² is CF₃; and R³ is C₁₋₆alkyl, such as C₁₋₃alkyl (e.g. methyl).

Within the invention there is provided another group of compounds of formula (I) (group C) wherein: R¹ is C₃₋₁₀cycloalkylmethyl, such as C₃₋₇cycloalkylmethyl (e.g. cyclopentylmethyl); R² is CF₃; and R³ is C₁₋₆alkyl, such as C₁₋₃alkyl (e.g. methyl).

Within the invention there is provided another group of compounds of formula (I) (group D) wherein: R¹ is A(CR⁴R⁵)ₙ; R² is CF₃; R³ is C₁₋₆alkyl, such as C₁₋₃alkyl (e.g. methyl); R⁴ and R⁵ are independently selected from H or methyl; A is selected from the group consisting of 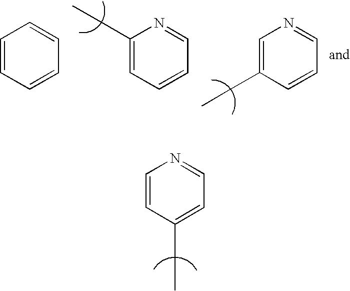
and A is unsubstituted or substituted by one or two R⁶; R⁶ is selected from the group consisting of halogen (e.g. F), C₁₋₃alkyl (e.g. methyl), C₁₋₃alkyl substituted by one to three fluorine atoms (e.g. CF₃). and C₁₋₃alkoxy (e.g. methoxy); and n is 0 to 2 (e.g. 0).

Within group D, there is provided a further group of compounds (group D1) wherein: R¹ is A(CR⁴R⁵)ₙ; R² is CF₃; R³ is methyl; R⁴ and R⁵ are both H; A is selected from the group consisting of 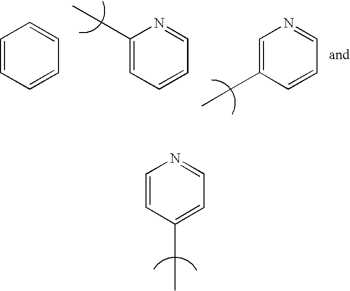
and A is unsubstituted or substituted by one or two R⁶; R⁶ is selected from the group consisting of fluorine, chlorine, methyl, CF₃ and methoxy; and n is 0 or 1.

Compounds of formula (I) and salts and solvates thereof are described in PCT publication No. WO02/096885, published 5 Dec. 2002 and U.S. application Ser. No. 10/477,547, published 2 Sep. 2004. The disclosures of these references are incorporated herein by reference in their entirety. Compounds of formula (I) may be prepared by any method described in WO 02/096885, U.S. application Ser. No. 10/477,547 and equivalent patent applications.

In a further embodiment, the present invention provides compounds of formula (I) and pharmaceutically acceptable salts or solvates thereof, for use in the preparation of a medicament for the treatment of schizophrenic disorders as defined above.

In another embodiment, the present invention provides a method for the treatment of schizophrenia, delusional disorders, affective disorders, autism or tic disorders, in particular chronic schizophrenic psychoses and schizoaffective psychoses, temporary acute psychotic disorders comprising administering a therapeutically effective amount of a compound of formula (I) or a pharmaceutically acceptable salts or solvates thereof.

In a one embodiment, the present invention provides a new use of compounds of formula (II) 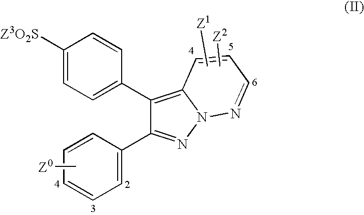
and pharmaceutically acceptable salts or solvates thereof, wherein
Z⁰ is selected from the group consisting of halogen, C₁₋₆alkyl, C₁₋₆-alkoxy, C₁₋₆-alkoxy substituted by one or more fluorine atoms, and O(CH₂)ₙNZ⁴Z⁵;
Z¹ and Z² are each the same or different and are independently selected from the group consisting of H, C₁₋₆-alkyl, C₁₋₆alkyl substituted by one or more fluorine atoms, C₁₋₆-alkoxy, C₁₋₆-hydroxyalkyl, SC₁₋₆-alkyl, C(O)H, C(O)C₁₋₆-alkyl, C₁₋₆-alkylsulphonyl, C₁₋₆-alkoxy substituted by one or more fluorine atoms, O(CH₂)ₙCO₂C₁₋₆alkyl, O(CH₂)ₙSC₁₋₆-alkyl, (CH₂)ₙNZ⁴Z⁵, (CH₂)ₙSC₁₋₆alkyl and C(O)NZ⁴Z⁵;
   with the proviso that when Z⁰ is at the 4-position and is halogen, then at least one of Z¹ and Z² is C₁₋₆alkylsulphonyl, C₁₋₆alkoxy substituted by one or more fluorine atoms, O(CH₂)ₙCO₂C₁₋₆alkyl, O(CH₂)ₙSC₁₋₆alkyl, (CH₂)ₙNZ⁴Z⁵, (CH₂)ₙSC₁₋₆alkyl or C(O)NZ⁴Z⁵;
Z³ is C₁₋₆alkyl or NH₂;
Z⁴ and Z⁵ are each the same or different and are independently selected from the group consisting of H, or C₁₋₆alkyl or, Z⁴ and Z⁵ together with the nitrogen atom to which they are bound, form a 4-8 membered saturated heterocyclic ring having 1 or 2 heteroatoms selected from N, O and S; and
n¹ is 1-4.

The term halogen is used to represent fluorine, chlorine, bromine or iodine.

The term ‘alkyl’ as a group or part of a group means a straight or branched chain alkyl group, for example a methyl, ethyl, n-propyl, i-propyl, n-butyl, s-butyl or t-butyl group.

Preferably, Z⁰ is at the 3- or 4-position of the phenyl ring, as defined in formula (I).

Preferably, Z¹ is at the 6-position of the pyridazine ring, as defined in formula (I).

Preferably, Z⁰ is F, C₁₋₃alkyl, C₁₋₃alkoxy, C₁₋₃alkoxy substituted by one or more fluorine atoms, or O(CH₂)₁₋₃NZ⁴Z⁵. More preferably Z⁰ is F, C₁₋₃alkoxy or C₁₋₃alkoxy substituted by one or more fluorine atoms.

Preferably, Z¹ is C₁₋₄alkylsulphonyl, C₁₋₄alkoxy substituted by one or more fluorine atoms, O(CH₂)₁₋₃CO₂C₁₋₄alkyl, O(CH₂)₁₋₃SC₁₋₄alkyl, (CH₂)₁₋₃NZ⁴Z⁵, (CH₂)₁₋₃SC₁₋₄alkyl or C(O)NZ⁴Z⁵ or, when Z⁰ is C₁₋₆alkyl, C₁₋₆alkoxy, O(CH₂)ₙNZ⁴Z⁵, may also be H. More preferably Z¹ is C₁₋₄alkylsulphonyl, C₁₋₄alkoxy substituted by one or more fluorine atoms or, when Z⁰ is C₁₋₆alkyl, C₁₋₆alkoxy, C₁₋₆alkoxy substituted by one or more fluorine atoms, or O(CH₂)ₙNZ⁴Z⁵, may also be H.

Preferably, Z² is H.

Preferably, Z³ is methyl or NH₂.

Preferably Z⁴ and Z⁵ are independently C₁₋₃alkyl or, together with the nitrogen atom to which they are attached, form a 5-6 membered saturated ring.

Preferably, n is 1-3,more preferably 1 or 2.

Within the invention there is provided one group of compounds of formula (I) (group A1) and pharmaceutically acceptable salts or solvates thereof, wherein: Z⁰ is F, C₁₋₃alkyl, C₁₋₃alkoxy, C₁₋₃alkoxy substituted by one or more fluorine atoms, or O(CH₂)ₙNZ⁴Z⁵; Z¹ is C₁₋₄alkylsulphonyl, C₁₋₄alkoxy substituted by one or more fluorine atoms, O(CH₂)ₙCO₂C₁₋₄alkyl, O(CH₂)ₙSC₁₋₄alkyl, (CH₂)ₙNZ⁴Z⁵, (CH₂)ₙSC₁₋₄alkyl or C(O)NZ⁴Z⁵ or, when Z⁰ is C₁₋₃alkyl, C₁₋₃alkoxy, C₁₋₃alkoxy subsbtuted by one or more fluorine atoms, or O(CH₂),NZ⁴Z⁵, may also be H; Z² is H; R³ is methyl or NH₂; Z⁴ and Z⁵ are independently C₁₋₃alkyl or, together with the nitrogen atom to which they are attached, form a 5-6 membered saturated ring; and n is 1-3.

Within group A, there is provided another group of compounds (group A2) and pharmaceutically acceptable salts or solvates thereof, wherein Z⁰ is F, methyl, C₁₋₂alkoxy, OCHF₂, or O(CH₂)ₙNZ⁴Z⁵; Z¹ is methylsulphonyl, OCHF₂, O(CH₂)ₙCO₂C₁₋₄alkyl, O(CH₂)ₙSCH₃, (CH₂)ₙNZ⁴Z⁵, (CH₂)ₙSCH₃ or C(O)NZ⁴Z⁵ or, when Z⁰ is methyl, C₁₋₂alkoxy, OCHF₂, or O(CH₂)ₙN(CH₃)₂, may also be H; Z² is H; Z³ is methyl or NH₂; Z⁴ and Z⁵ are both methyl or, together with the nitrogen atom to which they are attached, form a 5-6 membered saturated ring; and n is 1-2.

Within group A, there is provided a further group of compounds (group A3), and pharmaceutically acceptable salts or solvates thereof wherein Z⁰ is F, C₁₋₃alkoxy or C₁₋₃alkoxy substituted by one or more fluorine atoms; Z¹ is C₁₋₄alkylsulphonyl, C₁₋₄alkoxy substituted by one or more fluorine atoms or, when Z⁰ C₁₋₃alkoxy or C₁₋₃alkoxy substituted by one or more fluorine atoms, may also be H; Z² is H; and Z³ is methyl or NH₂.

Within groups A1, A2 and A3,Z⁰ is preferably at the 3- or 4-position of the phenyl ring and Z¹ is preferably at the 6-position of the pyridazine ring.

Compounds of formula (II) and pharmaceutically acceptable salts and solvates thereof are described in PCT publication No. WO 99/12930, published 18 Mar. 1999 and U.S. Pat. No. 6,451,794, US-A-2003-0040517 and US-A-2003-0008872. The disclosures of these references are incorporated herein by reference in their entirety. Compounds of formula (II) may be prepared by any method described in WO 99/12930, U.S. Pat. No. 6,451,794, US-A-2003-0040517 and US-A-2003-0008872 and equivalent patent applications.

In a further embodiment, the present invention provides compounds of formula (II) and pharmaceutically acceptable salts or solvates thereof, for use in the preparation of a medicament for the treatment of schizophrenic disorders as defined above.

In another embodiment, the present invention a method for the treatment of schizophrenia, delusional disorders, affective disorders, autism or tic disorders, in particular chronic schizophrenic psychoses and schizoaffective psychoses, temporary acute psychotic disorders comprising administering a therapeutically effective amount of a compound of formula (II) or a pharmaceutically acceptable salts or solvates thereof.

In one embodiment the present invention provides a new use of compounds of formula (III) 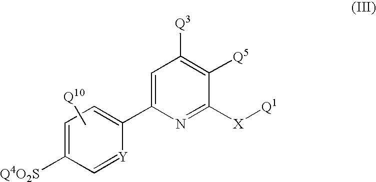
and pharmaceutically acceptable salts or solvates thereof, wherein:
X is selected from the group consisting of oxygen or NQ²;
Y is selected from the group consisting of CH or nitrogen;
Q¹ is selected from the group consisting of H, C₁₋₆alkyl, C₁₋₂alkyl substituted by one to five fluorine atoms, C₁₋₃alkylOC₁₋₃alkyl, C₃₋₆alkenyl, C₃₋₆alkynyl, C₃₋₁₀cycloalkylCO₀₋₆alkyl, C₄₋₇cycloalkyl substituted by C₁₋₃alkyl or C₁₋₃-alkoxy, C₄₋₁₂bridged cycloalkyl, A(CR⁶R⁷)ₙ and B(CR⁶R⁷)ₙ;
Q² is selected from the group consisting of H and C₁₋₆alkyl; or
Q¹ and Q² together with the nitrogen atom to which they are attached form a 4-8 membered saturated heterocyclic ring such as a pyrrolidine, morpholine or piperidine ring, or a 5-membered heteroaryl ring which is unsubsttuted or substituted by one R⁸;
Q³ is selected from the group consisting of C₁₋₅alkyl and C₁₋₂alkyl substituted by one to five fluorine atoms;
Q⁴ is selected from the group consisting of C₁₋₆alkyl, NH₂ and R⁹CONH;
Q⁵ is selected from the group consisting of hydrogen, C₁₋₃alkyl, C₁₋₂alkyl substituted by one to five fluorine atoms, C₁₋₃alkylO₂C, halogen, cyano, (C₁₋₃alkyl)₂NCO, C₁₋₆alkylS and C₁₋₃alkylO₂S;
Q⁶ and Q7 are independently selected from H or C₁₋₆alkyl;
A¹ is an unsubstituted 5- or 6-membered heteroaryl or an unsubstituted 6-membered aryl, or a 5- or 6-membered heteroaryl or a 6-membered aryl substituted by one or more R⁸;
Q⁸ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and ClalkylSO₂;
B¹ is selected from the group consisting of 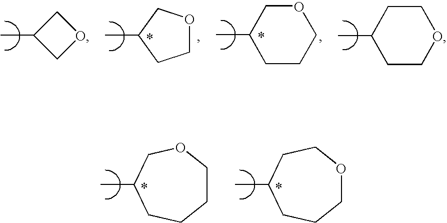
and where 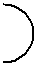
defines the point of attachment of the ring;
Q⁹ is selected from the group consisting of H, C₁₋₆alkyl, C₁₋₆alkoxy, C₁₋₆-alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆-alkyl and C₁₋₆alkyl CONHC₁₋₆alkyl;
Q¹⁰ is selected from the group consisting of H and halogen; and
n is 0 to 4;

The term ‘halogen’ is used to represent fluorine, chlorine, bromine or iodine.

The term ‘alkyl’ as a group or part of a group means a straight or branched chain alkyl group, for example a methyl, ethyl, n-propyl, i-propyl, n-butyl, s-butyl or t-butyl group.

The term ‘saturated heterocyclic’ means a saturated ring containing at least one atom other than carbon.

The term ‘5-membered heteroaryl’ means a heteroaryl selected from the following:

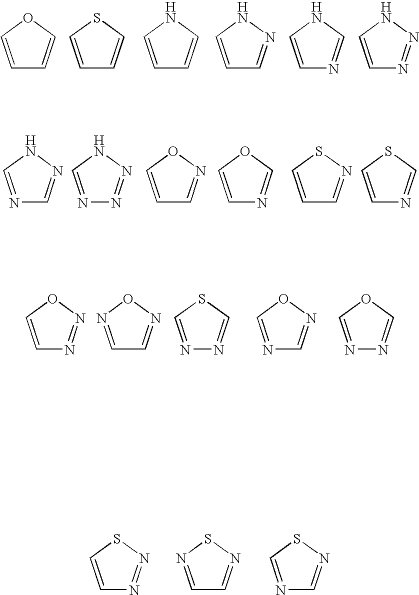

The term ‘6- membered heteroaryl’ means a heteroaryl selected from the following:

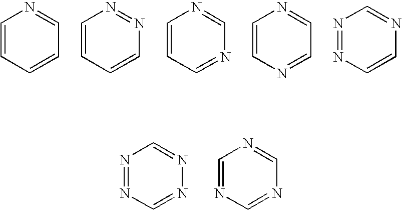

The term ‘6-membered aryl’ means:

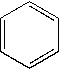

Compound of formula (III) may be a compound of formula (IIIC) 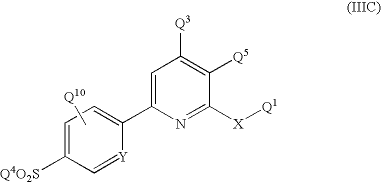
and pharmaceutically acceptable salts or solvates thereof, wherein
X is selected from the group consisting of oxygen or NR²;
Y is selected from the group consisting of CH or nitrogen;
Q¹ is selected from the group consisting of H, C₁₋₆-alkyl, C₁₋₂alkyl substituted by one to five fluorine atoms, C₁₋₃-alkylOC₁₋₃alkyl, C₃₋₆alkenyl, C₃₋₆alkynyl, C₃₋₁₀cycloalkylC₀₋₆alkyl, C₄₋₁₂bridged cycloalkyl, A(CQ⁸Q⁷)ₙ and B(CQ⁶Q⁷)ₙ;
Q² is selected from the group consisting of H and C₁₋₆alkyl; or
Q¹ and Q² together with the nitrogen atom to which they are attached form a 4-8 membered saturated heterocyclic ring such as a pyrrolidine, morpholine or piperidine ring;
Q³ is selected from the group consisting of C₁₋₅-alkyl and C₁₋₂alkyl substituted by one to five fluorine atoms;
Q⁴ is selected from the group consisting of C₁₋₆-alkyl, NH₂ and Q⁹CONH;
Q⁵ is selected from the group consisting of hydrogen, C₁₋₃alkyl, C₁₋₂alkyl substituted by one to five fluorine atoms, halogen, cyano, (C₁₋₃-alkyl)₂NCO, C₁₋₃-alkylS and C₁₋₃-alkylO₂S;
Q⁶ and Q⁷ are independently selected from H or C₁₋₆alkyl;
A¹ is an unsubstituted 5- or 6-membered heteroaryl or an unsubstituted 6-membered aryl, or a 5- or 6-membered heteroaryl or a 6-membered aryl substituted by one or more Q⁸;
Q⁸ is selected from the group consisting of halogen, C₁₋₆alkyl, C₁₋₆alkyl substituted by one more fluorine atoms, C₁₋₆alkoxy, C₁₋₆-alkoxy substituted by one or more F, NH₂SO₂ and C₁₋₆alkylSO₂;
B¹ is selected from the group consisting of 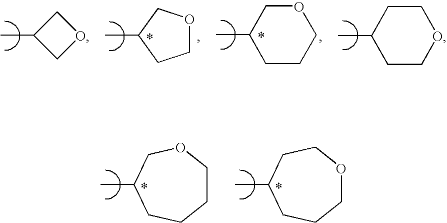
and where 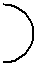
defines the point of attachment of the ring;
Q⁹ is selected from the group consisting of H, C₁₋₆-alkyl, C₁₋₆-alkoxy, C₁₋₆-alkylOC₁₋₆-alkyl, phenyl, HO₂CC₁₋₆-alkyl, C₁₋₆alkylOCOC₁₋₆alkyl, C₁₋₆-alkylOCO, H₂NC₁₋₆alkyl, C₁₋₆-alkylOCONHC₁₋₆alkyl and C₁₋₆alkyl CONHC₁₋₆alkyl;
Q¹⁰ is selected from the group consisting of H and halogen; and
n is 0 to 4.

Compound of formula (III) may be a compound of formula (IIID) 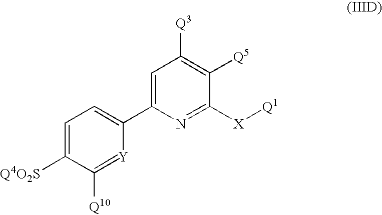
and pharmaceutically acceptable salts or solvates thereof, wherein all substituents are as for a compound of formula (III) defined hereinabove.

Compound of formula (III) may be a compound of formula (IIIE) 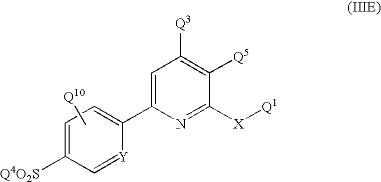
and pharmaceutically acceptable salts or solvates thereof, wherein
X is selected from the group consisting of oxygen or NQ²;
Y is selected from the group consisting of CH or nitrogen;
Q¹ is selected from the group consisting of H, C₁₋₆alkyl, C₁₋₂alkyl substituted by one to five fluorine atoms, C₁₋₃alkylOC₁₋₃alkyl, C₃₋₆alkenyl, C₃₋₆alkynyl, C₃₋₁₀-locycloalkylC₀₋₆alkyl, C₄₋₇cydoalkyl substituted by C₁₋₃alkyl or C₁₋₃alkoxy, C₄₋₁₂bridged cycloalkyl, A(CR⁶R⁷)ₙ and B(CR⁶R⁷)ₙ;
Q² is selected from the group consisting of H and C₁₋₆alkyl; or
Q¹ and Q² together with the nitrogen atom to which they are bound form a 4-8 membered saturated heterocyclic ring or a 5-membered heteroaryl ring heteroaryl ring is unsubstituted or substituted by one R⁸; Q³ is selected from the group consisting of C₁₋₅alkyl and C₁₋₂alkyl substituted by one to five fluorine atoms;
Q⁴ is selected from the group consisting of C₁₋₆alkyl, NH₂ and R⁹CONH;
Q⁵ is selected from the group consisting of hydrogen, C₁₋₃alkyl, C₁₋₂alkyl substituted by one to five fluorine atoms, C₁₋₃alkylO₂C, halogen, cyano, (C₁₋₃alkyl)₂NCO, C₁₋₃alkylS and C₁₋₃alkylO₂S;
Q⁶ and Q⁷ are independently H or C₁₋₆alkyl;
A¹ is selected from the group consisting of unsubstituted 5- or 6-membered heteroaryl unsubstituted 6-membered aryl, 5- or 6-membered heteroaryl substituted by one or more R⁸; and 6-membered aryl substituted tby one or more R⁸;
Q⁸ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B¹ is a ring selected from the group consisting of 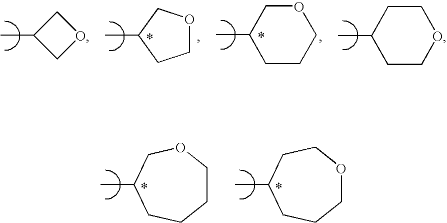
and where 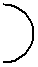
defines the point of aftachment of the ring;
Q⁹ is selected from the group consisting of H, C₁₋₆-alkyl, C₁₋₆-alkoxy, C₁₋₆-alkylOC₁₋₆alkyl, phenyl, HO₂CC₁₋₆alkyl, C₁₋₆-alkylOCOC₁₋₆alkyl, C₁₋₆-alkylOCO, H₂NC,-alkyl, C₁₋₆-alkylOCONHC₁₋₆-alkyl and C₁₋₆-alkylCONHC₁₋₆alkyl;
Q¹⁰ is selected from the group consisting of H and halogen; and
n is 0 to 4.

In another aspect of the invention Y is carbon.

In another aspect of the invention Q¹ is selected from the group consisting of, C₁₋₆-alkyl, C₃₋₁₀cycloalkylC₀₋₆alkyl, C₅₋₆cycloalkyl substituted by C₁₋₂alkyl or C₁₋₂alkoxy, C₁₋₃alkylOC₁₋₃-alkyl and C₁₋₂alkyl substituted by one to five fluorine atoms.

Representative examples of Q¹ include cyclohexylmethyl, cyclohexyl, n-butyl, n-pentyl, cyclopentyl, 2-methylpropyl, 2,2-dimethylpropyl, 2,2,2-trifluoroethyl, 2-methoxyethyl and ethyl.

Further representative examples of Q¹ include 1-methylethyl, 1-ethylpropyl, cycloheptyl, cis-4methylcyclohexyl, trans-4-methylcyclohexyl, cyclobutyl, cyclopentanemethyl, and trans-4-(ethoxy)cyclohexyl.

In another aspect of the invention Q¹ is selected from the group consisting of A¹(CQ⁶Q⁷)ₙ and B¹(CQ⁶Q⁷)ₙ.

Further representative examples of Q¹ include benzyl, 4-chlorobenzyl, 2-furylmethyl, 4-methylphenyl, 4-fluorophenyl, 4-methoxyphenyl, 3-pyridyl, 2-chlorophenyl, 3,5-difluorobenzyl, 3-pyridylmethyl, 2-methylbenzyl, 2-chlorobenzyl, (S)-α-methylbenzyl, (R)-α-methylbenzyl, 6-methylpyridin-3-yl, 4-methoxybenzyl, 4-fluorobenzyl, 2-(5-methylfuryl)methyl, 4-methylbenzyl, 4-pyridylmethyl, 2-pyridylmethyl, 2-(6-methylpyridine)methyl, 2-thiophenylmethyl, 4-pyranylmethyl, 2-tetrahydrofurylmethyl, 2-(5-methylpyrazine)methyl and 4-ethoxybenzyl.

Further representative examples of Q¹ include 1H-imidazol-2-ylmethyl, 1H-pyrazol-4-ylmethyl, (1-methyl-1H-imidazol-2-yl)methyl, (3-methyl-1H-pyrazol4-yl)methyl, (1-methyl-1H-pyrazol-3-yl)methyl, (1-methyl-1H-pyrazol-4-yl)methyl, (3-methyl-1H-pyrazol-5-yl)methyl, (1-methyl-1H-pyrazol-5-yl)methyl, (1-methyl-1H-1,2,4-triazol-5-yl)methyl, (5-methyl-3-isoxazolyl)methyl, tetrahydro-2H-pyran-4-yl, tetrahydro-2H-pyran-4-ylmethyl, (6-methyl-3-pyridyl)methyl, 2-pyrazinylmethyl, (2-methyl-1H-imidazol-4-yl)methyl, (4-methyl-1H-imidazol-5-yl)methyl, (4-methyl-1H-imidazol-2-yl)methyl, (1-ethyl-1H-imidazol-2-yl)methyl, (1,3-dimethyl-1H-pyrazol-4-yl)methyl, (1,5-dimethyl-1H-pyrazol-4-yl)methyl, (3-methyl-5-isothiazolyl)methyl, (4-methyl-1,3-thiazol-2-yl)methyl, (3-methyl-4-isothiazolyl)-methyl, [1-(fluoromethyl)-1H-pyrazol-4-yl]methyl, (2-methyl-3-pyridyl)methyl, (6-methyl-3-pyridyl)methyl, (1-methyl-1H-imidazol-2-yl)methyl, (5-chloro-2-pyridyl)methyl, 1H-imidazol-2-ylmethyl, 4-ethoxyphenyl, 3-chloro-4-methylphenyl, (5-chloro-2-pyridyl)methyl, (6-methyl-3-pyddyl)methyl, 2-methyl-3-pyridyl, 6-methyl-2-pyridyl, 2-pyrazinylmethyl, 2,6-dimethyl-3-pyridyl, 3,4-dichlorobenzyl, 5-chloro-3-pyridyl, 6-chloro-3-pyridazinyl, 3,5-dichlorobenzyl, 2-carboxyphenyl, (5-methyl-2-pyridyl)methyl, 4-chloro-3-(trifluoro-methyl)benzyl, (5-bromo-2-pyridyl)methyl, (4-bromo-4-pyridyl)methyl, (3-methyl-4-isoxazolyl)methyl, 5-pyrimidinylmethyl, (3-methyl-1,2,4-oxadiazol-5-yl)methyl, (5-methyl-1,2,4-oxadiazol-3-yl)methyl and (1-ethyl-1H-1,2,4-triazol-5-yl)methyl.

In another aspect of the invention Q¹ is selected from the group consisting of C₃₋₆alkenyl and C₃₋₆alkynyl.

Further representative examples of Q¹ include propargyl and allyl.

In another aspect of the invention Q² is H or C₁₋₂alkyl.

Representative examples of Q² include H, methyl and ethyl.

In another aspect of the invention Q³ is CHF₂, CH₂F, CF₃or C₁₋₄alkyl.

Representative examples of Q³ include CF₃, CH₃ and ethyl.

Further representative examples of Q³ include CH₂F.

In another aspect of the invention Q⁴ is C₁₋₆alkyl, such as C₁₋₃alkyl.

Representative examples of Q⁴ include CH₃.

In another aspect of the invention Q⁴ is NH₂.

Further representative examples of Q⁴ include NH₂.

In another aspect of the invention Q⁵ is hydrogen or C₁₋₃alkyl.

Representative examples of Q⁵ include H or CH₃.

In another aspect of the invention R⁵ is CN, halogen or CO₂Et.

Further representative examples of Q⁵ indude CN, F, Cl, CO₂Et.

In another aspect of the invention Q⁶ and Q⁷ are independently selected from H or methyl.

In another aspect Q⁶ and Q⁷ are both H.

In another aspect of the invention A¹ is selected from the group consisting of 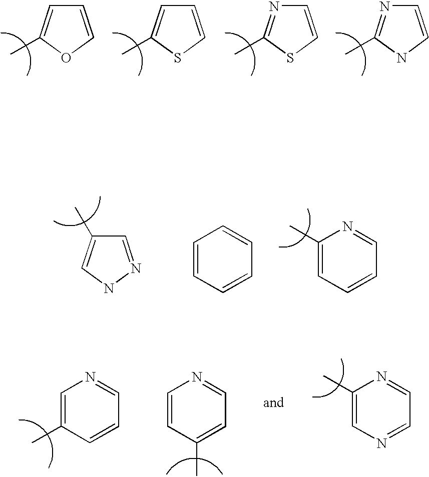
where 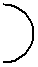
defines the point of attachment of the ring and A¹ is unsubstituted or substituted by one or two Q⁸.

In another aspect of the invention Al is selected from the group consisting of 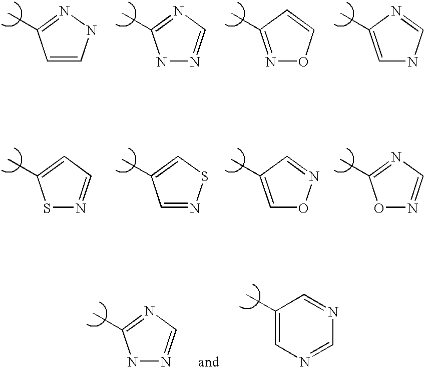
where 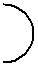
defines the point of attachment of the ring

In another aspect of the invention Q⁸ is selected from the group consisting of halogen, C₁₋₃alkyl, C₁₋₄alkyl substituted by one to three fluorine atoms (e.g. CF₃), and C₁₋₃alkoxy.

Representative examples of Q⁸ include F, Cl, CH₃, methoxy and ethoxy.

Further representative examples of Q⁸ include ethyl, fluoromethyl, CF₃ and Br.

Representative examples of B¹ include

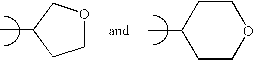

In another aspect of the invention Q⁹ is selected from the group consisting of C₁₋₆alkyl (e.g. ethyl), phenyl and aminomethyl.

In another aspect of the invention Q¹⁰ is H.

In another aspect of the invention in compounds of formula (III), (IIIC) and (IIID) n is 0 to 2 (e.g. 1) or in compounds of formula (IIIE) n is 1 or 2.

In another aspect the invention provides a compound of formula (III) or a pharmaceutically acceptable salt or solvate thereof in which:
X is oxygen;
Y is CH;
Q¹ is A¹ (CR⁶R⁷)ₙ;
Q³ is selected from the group consisting of C₁₋₅alkyl and C₁₋₂alkyl substituted by one to five fluorine atoms;
Q⁴ is C₁₋₆alkyl;
Q⁵ is selected from the group consisting of hydrogen, C₁₋₃alkyl, C₁₋₂alkyl substituted by one to five fluorine atoms, C₁₋₆-alkylO₂C, halogen, and C₁₋₃alkylS;
A¹ is an unsubstituted 5- or 6-membered heteroaryl or an unsubstituted 6-membered aryl, or a 5- or 6-membered heteroaryl or a 6-membered aryl substituted by one or more R⁸;
Q⁸ is selected from the group consisting of halogen, C₁₋₆-alkyl, C₁₋₆-alkyl substituted by one more fluorine atoms, C₁₋₆-alkoxy, and C₁₋₆-alkoxy substituted by one or more F;
Q¹⁰ is selected from the group consisting of H and halogen; and
n is 0.

Compounds of formula (III) and pharmaceutically acceptable salts and solvates thereof are described in PCT publication No. WO 2004/024691, published 25 Mar. 2004. The disclosures of these references are incorporated herein by reference in their entirety. Compounds of formula (III) may be prepared by any method described in WO 2004/024691 and equivalent patent applications.

In a further embodiment, the present invention provides compounds of formula (III) and pharmaceutically acceptable salts or solvates thereof, for use in the preparation of a medicament for the treatment of schizophrenic disorders as defined above.

In another embodiment, the present invention a method for the treatment of schizophrenia, delusional disorders, affective disorders, autism or tic disorders, in particular chronic schizophrenic psychoses and schizoaffective psychoses, temporary acute psychotic disorders comprising administering a therapeuucally effective amount of a compound of formula (III) or a pharmaceutically acceptable salts or solvates thereof.

In one embodiment of the present invention provides the use of a compound of formula selected from the following group consisting of:
2-(4-fluorophenoxy)-4-[4-(methylsulfonyl)phenyl]-6-(trifluoromethyl)pyrimidine;
2-(4-methoxyphenoxy)-4-[4-(methylsulfonyl)phenyl]-6-tifluoromethyl)pydmidine;
2-butoxy-4-[4-(methylsulfonyl)phenyl]-6-(trifluoromethyl)pyrimidine;
2-[(5-chloropyridin-3-yl)oxy]-4-[4-(methylsulfony)phenyl]-6-(trifluoromethyl)pyrimidine;
2-(cyclohexyloxy)-4-[4-(methylsulfonyl)phenyl]-6-(trifluoromethyl)pyrimidine;
3-(4-methylsulfonyl-phenyl)-2-(4-methoxy-phenyl)pyrazolo[1,5-b]pyridazine;
6-difluoromethoxy-2-(4-fluoro-phenyl)-3-(4-methylsulfonyl-phenyl)-pyrazolo[1,5-b]-pyridazine;
2-(4-ethoxy-phenyl)-3-(4-methylsulfonyl-phenyl)-pyrazolo[1,5-b]pyridazine;
2-(4-fluoro-phenyl)-6-methylsulfonyl-3-(4-methylsulfonyl-phenyl)-pyrazolo[1,5-b]pyridazine;
2-(4-difluoromethoxy-phenyl)-3-(4-methylsulfonyl-phenyl)-pyrazolo[1,5-b]pyridazine;
4-[2-(4-ethoxy-phenyl)-pyrazolo[1,5-b]pyridazin-3-yl]-benzenesulfonamide;
6-difluoromethoxy-2-(3-fluoro-phenyl)-3-(4-methylsulfonyl-phenyl-pyrazolo[1,5-b]pyridazine;
3-(4-methanesulfonyl-phenyl)-2-(4-methoxy-phenyl)-pyrazolo[1,5-b]pyridazine;
6-difluoromethoxy-2-(4-fluoro-phenyl)-3-(4-methanesulfonyl-phenyl)-pyrazolo[1,5-b]pyridazine;
2-(4-ethoxy-phenyl)-3-(4-methanesulfonyl-phenyl)-pyrazolo[1,5-b] pyridazine;
2-(4-fluoro-phenyl)-6-methanesulfonyl-3-(4-rethanesulfonyi-phenyl)-pyrazolo[1,5-b]pyridazine;
2-(4-difluoromethoxy-phenyl)-3-(4-methanesulfonyl-phenyl)-pyrazolo[1,5-b]pyridazine;
4-[2-(4-ethoxy-phenyl)-pyrazolo[1,5-b]pyridazin-3-yl]-benzenesulfonamide;
6-difluoromethoxy-2-(3-fluoro-phenyl)-3-(4-methanesulfony5-phenyl)-pyrazolo[1,5-b]pyridazine
4-ethyl-6-[4-(methylsulfonyl)phenyl]-N-(tetrahydro-2H-pyran-4-ylmethyl)-2-pyridinamine; 4-methyl-N-[(1-methyl-1H-pyrazol-4-yl)methyl]-6-[4-(methylsulfonyl)phenyl]-2-pyridinamine;
N-[(1,5-dimethyl-1H-pyrazol-4-yl)methyl]4-methyl-6-[4-(methylsulfonyl)phenyl]-2-pyridinamine;
N-[(1,3-dimethyl-1H-pyrazol-4-yl)methyl]4-methyl-6-[4-(methylsulfonyl)phenyl]-2-pyridinamine;
4-(6{[(1,3-dimethyl-1H-pyrazol-4-yl)methyl]amino}4-ethyl-2-pyridinyl)benzene-sulfonamide;
N-[(1,3-dimethyl-1H-pyrazol-4-yl)methyl]-6-[4-(methylsulfonyl)phenyl]-4-(trifluoromethyl)-2-pyridinamine;
N-[(1,5-dimethyl-1H-pyrazol-4-yl)methyl]-6-[4-(methylsulfonyl)phenyl]-4-(trifluoromethyl)-2-pyridinamine;
4-{4-methyl-6-[(tetrahydro-2H-pyran-4-yimethyl)amino]-2-pyridinyl}benzenesulfonamide;
4-methyl-N-[(1-methyl-1H-pyrazol-3-yl)methyl]-6-[4-(methylsulfonyl)phenyl]-2-pyridinamine;
N-(cyclohexylmethyl)-6-[4-(methylsulfonyl)phenyl]-4-(trifluoromethyl)-2-pyridinamine;
N-cyclohexyl-6-[4-(methylsulfonyl)phenyl]-4-(trifluoromethyl)-2-pyridinamine;
2-[4-(methylsulfonyl)phenyl]-6-[(2-pyridinylmethyl)oxy]-4-(trifluoromethyl)pyridine;
4-methyl-N-[(3-methyl-4-isoxazolyl)methyl]-6-[4-(methylsuffonyl)pheny]-2-pyridinamine;
6-[4-(methylsufonyl)phenyl]-N-(2-pyridinylmethyl)-4-(tdfluoromethyl)-2-pyridinamine;
N-cycloheptyl-6-[4-(methylsulfonyl)phenyl]-4-(trifluoromethyl)-2-pyridinamine;
N-(cis4-methylcyclohexyl)-6-[4-(methylsulfonyl)phenyl]-4-(trifluoromethyl)-2-pyridinamine;
N-(1-ethylpropyl)-6-[4-(methylsulfonyl)phenyl]-4-(trifluoromethyl)-2-pyridinamine;
N-[(3-methyl-1,2,4-oxadiazol-5-yl)methy]-6-[4-(methylsulfonyl)phenyl]-4-(trifluoromethyl)-2-pyridinamine;
N-[(5-methyl-1,2,4-oxadiazol-3-yl)methyl]-6-[4-(methylsulfonyl)phenyl]-4-(trifluoromethyl)-2-pyridinamine;
4-methyl-N-[(1-methyl-1H-pyrazol-5-yl)methyl]-6-[4-(methylsulfonyl)phenyl]-2-pyridinamine;
N-(cyclopentylmethyl)-6-[4-(methylsulfonyl)phenyl]-4-(trifluoromethyl)-2-pyridinamine;
N-[(1-ethyl-1H-1,2,4-triazol-5-yl)methyl]-4-methyl-6-[4-(methylsulfonyl)phenyl]-2-pyridinamine;
4-ethyl-6-[4-(methylsulfonyl)phenyl]-2-[(2-pyridinylmethyl)amino]-3-pyridinecarbonitrile;
4-ethyl-2-{[(5-methyl-2-pyridinyl)methyl]amino}-6-[4-(methylsulfonyl)phenyl]-3-pyridinecarbonitrile;
4-ethyl-2-{[(6-methyl-3-pyridinyl)methyl]amino}-6-[4-(methylsulfonyl)phenyl]-3-pyridinecarbonitrile;
4-ethyl-2-{[(1-methyl-1H-pyrazol4-yl)methyl]amino}-6-[4-(methylsulfonyl)phenyl]-3-pyridinecarbonitrile;
4-ethyl-{6-[4-(methylsulfonyl)phenyl]-2-[(4-methyl-1,3-thiazol-2-yl)methyl]amino}-3-pyridinecarbonitrile;
4-ethyl-6-[4-(methylsulfonyl)phenyl]-2-[(2-pyddinylmethyl)oxy]-3-pyridinecarbonitrile;
4-ethyl-N-[(1-ethyl-1H-1,2,4-triazol-5-yl)methyl]-6-[4-(methylsulfonyl)phenyl]-2-pyridinamine;
4-ethyl-2-{[(6-methyl-3-pyridinyl)methyl]oxy}-6-[4-(methylsulfonyl)phenyl]-3-pyridinecarbonitrile;
6-[4-(methylsulfonyl)phenyl]-N-[(1-methyl-1H-1,2,4-triazol-5-yl)methyl]-4-(trifluoromethyl)-2-pyridinamine;
   and pharmaceutically acceptable salts and solvates thereof, for use in the treatment of schizophrenic disorders as defined above and the preparation of a medicament for the treatment of schizophrenic disorders

In a particular embodiment of the present invention the compound is selected from the group consisting of: 2-(4-ethoxy-phenyl)-3-(4-methylsulfonyl-phenyl)-pyrazolo[1,5-b]-pyridazine; 2-butoxy-4-[4-(methylsulfonyl)phenyl]6-(trifluoromethyl)pyrimidine; N-cyclo-hexyl-6-[4-(methylsulfonyl)phenyl]-4-(trifluoromethyl)-2-pyridinamine; 2-[4-(methylsulfonyl-)phenyl]-6-[(2-pyridinylmethyl)oxy]-4-(trifluoromethyl)pyridine; 4-methyl-N-[(1-methyl-1H-pyrazol-4-yl)methyl]-6-[4-(methylsulfonyl)phenyl]-2-pyridinamine; 3-(4-methanesulfonyl-phenyl)-2-(4-methoxy-phenylypyrazolo[1,5-b]pyridazine; 6-difluoromethoxy-2-(4-fluoro-phenyl)-3-(4-methanesulfonyl-phenyl)-pyrazolo[1,5-b]pyridazine; 2-(4-ethoxy-phenyl)-3-(4-methanesulfonyl-phenyl)-pyrazolo[1,5-b]pyridazine; 2-(4-fluoro-phenyl)-4-methane-sulfonyl-3-(4-methanesulfonyl-phenyl)pyrazolo[1,5-b]pyridazine; 2-(4-difluoromethoxy-phenyl)-3-(4-methanesulfonyl-phenyl)-pyrazolo[1,5-b]pyridazine; 4-[2-(4-ethoxy-phenyl)-pyrazolo[1,5-b]pyridazin-3-yl]-benzenesulfonamide; 6-difluoromethoxy-2-(3-fluoro-phenyl)-3-(4-methanesulfonyl-phenyl)pyrazolo[1,5-b]pyridazine; or a pharmaceutically acceptable salt or solvate thereof.

Conveniently, compounds of formula (I), (II) and (III) of the invention are isolated following work-up in the form of the free base. Pharmaceutically acceptable acid addition salts of the compounds of the invention may be prepared using conventional means.

It is intended that reference to particular compounds herein be interpreted to mean that the pharmaceutically acceptable salts, solvates and prodrugs of those compounds may also be employed.

Typically, a pharmaceutical acceptable salt may be readily prepared by using a desired acid or base as appropriate. The salt may precipitate from solution and be collected by filtration or may be recovered by evaporation of the solvent.

Suitable addition salts are formed from acids which form non-toxic salts and examples are hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, piruvate, oxalate, oxaloacetate, trifluoroacetate, saccharate, benzoate, methansulphonate, ethanesulphonate, benzenesulphonate, p-toluensulphonate, methanesulphonic, ethanesulphonic, p-toluenesulphonic, and isethionate.

In addition, prodrugs are also included within the context of this invention.

As used herein, the term uprodrug¹ means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987,and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I), (II) and (III) in vivo when such prodrug is administered to a patient. Prodrugs are generally prepared by modifying functional groups in a way such that the modification is deaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of structure (I), (II) and (III).

With regard to stereoisomers, the compounds of structure (I), (II) and (III) may have one or more asymmetric carbon atom and may occur as recemates, racemic mixtures and as individual enantiomers or diastereomers. All such isomeric forms are included within the present invention, including mixtures thereof.

Furthermore, the invention concerns the use of COX-2 inhibitors of formula (I), (II) and (III) in combination with neuroleptics for the treatment of schizophrenic disorders such as schizophrenia, delusional disorders, affective disorders, autism or tic disorders, in particular chronic schizophrenic psychoses and schizoaffective psychoses, temporary acute psychotic disorders.

The invention is also directed to a novel kit-of-parts that is suitable for use in the treatment of schizophrenic disorders as above defined, the kit comprising a first dosage form comprising a neuroleptic and a second dosage form comprising a COX-2 inhibitor, for simultaneous, separate or sequential administration.

The compounds of formula (I), (II) and (III) and their pharmaceutically acceptable salts and solvates are conveniently administered in the form of pharmaceutical compositions. Such compositions may conveniently be presented for use in conventional manner in admixture with one or more physiologically acceptable carriers or excipients.

The compounds of formula (I), (II) and (III), and their pharmaceutically acceptable derivatives may be formulated for administration in any suitable manner. They may, for example, be formulated for topical administrabon or administration by inhalation or, more preferably, for oral, transdermal or parenteral administration. The pharmaceutical composition may be in a form such that it can effect controlled release of the compounds of formula (I), (II) and (III), and their pharmaceutically acceptable derivatives.

For oral administration, the pharmaceutical composition may take the form of, for example, tablets (induding sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehides and may contain formulatory agents such as suspending, stabilising and/or dispersing agents. For administration by injection these may take the form of a unit dose presentation or as a multidose presentation preferably with an added preservative.

Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

As stated above, the compounds of the invention may also be used in combination with other therapeutic agents. The invention thus provides, in a further aspect, a combination comprising a compound of formula (I), (II) or (III) or a pharmaceutically acceptable derivative thereof together with a further therapeutic agent.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

A proposed daily dosage of a compound of formula (I), (II) and (III) for the treatment of man is 0.01 mg/kg to 500 mg/kg, such as 0.05 mg/kg to 100 mg/kg, e.g. 0.8-3.0 mg/kg, which may be conveniently administered in 1 to 4 doses. The precise dose employed will depend on the age and condition of the patient and on the route of administration. Thus, for example, a daily dose of 0.25 mg/kg to 10 mg/kg may be suitable for systemic administration.

In a particular embodiment of the present invention, compounds of formula (I), (II) and (III) are used in the form of tablets for oral administration.

According to a further embodiment of the present invention, the COX-2 inhibitor of the present invention is used in combination with a neuroleptic drug for the manufacture of a medicament for the treatment of schizophrenic disorders as defined above.

Combinations can also include a mixture of one or more COX-2 inhibitors of the present invention or a mixture of one COX-2 inhibitor of the present invention with another COX-2 inhibitor, for example, available on the market (Celebrex®) or generally known as COX-2 inhibitor with one or more neuroleptic agents, mood stabilisers or antimanic.

In a further particular embodiment of the present invention, the combination of a COX-2 inhibitor with a neuroleptic drug is useful for the treatment of schizophrenia.

Both classical and atypical neuroleptics can be used for the add-on use according to the invention, in particular atypical neuroleptics.

Examples of neuroleptic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene ; thienobenzodiazepines; dibenzodiazepines; benzisoxazoles; dibenzothiazepines; imidazolidinones ; benziso-thiazolyl-piperazines; triazine such as lamotrigine; dibenzoxazepines, such as loxapine; dihydroindolones, such as molindone; aripiprazole; and derivatives thereof that have antipsychotic activity.

Examples of neuroleptic drugs that may be selected for use in the present invention are shown in Table 1.

TABLE 1
Neuroleptic drugs

| | | | | Dosage | |
|---|---|---|---|---|---|
| Common | | Route of | | Range and | |
| Name | Trade Name | Administration | Form | (Median)^{a} | |
| Clozapine | CLOZARIL | oral | tablets | 12.5-900 | mg/day |
| | | | | (300-900 | mg/day) |
| Olanzapine | ZYPREXA | oral | tablets | 5-25 | mg/day |
| | | | | (10-25 | mg/day) |
| Ziprasidone | GEODON | oral | capsules | 20-80 | mg/twice a day |
| | | | | (80-160 | mg/day) |
| Risperidone | RISPERDAL | oral | solution tablets | 2-16 | mg/day |
| | | | | tablets | |
| | | | | (4-12 | mg/day) |
| Quetiapine | SEROQUEL | oral | tablets | 50-900 | mg/day |
| fumarate | | | | (300-900 | mg/day) |
| Sertindole | SERLECT | | | (4-24 | mg/day) |
| Amisulpride | | | | | |
| Haloperidol | HALDOL | oral | tablets | 1-100 | mg/day |
| | | | | (1-15 | mg/day) |
| Haloperidol | HALDOL | parenteral | injection | | |
| Decanoate | Decanoate | | | | |
| Haloperidol lactate | HALDOL | oral | solution | | |
| | INTENSOL | parenteral | injection | | |
| Chlorpromazine | THORAZINE | rectal | suppositories | 30-800 | mg/day |
| | | oral | capsules | (200-500 | mg/day) |
| | | | solution | | |
| | | | tablets | | |
| | | parenteral | injection | | |
| Fluphenazine | PROLIXIN | | | 0.5-40 | mg/day |
| | | | | (1-5 | mg/day) |
| Fluphenazine | PROLIXIN | parenteral | injection | (about one-half the | |
| decanoate | Decanoate | | | dosage shown for | |
| | | | | oral) | |
| Fluphenazine | PROLIXIN | parenteral | injection | (same as above | |
| enanthate | | | | | |
| Fluphenazine | PROLIXIN | oral | elixer | | |
| hydrochloride | | | solution | | |
| | | parenteral | injection | | |
| Thiothixene | NAVANE | oral | capsules | 6-60 | mg/day |
| | | | | (8-30 | mg/day) |
| Thiothixene | NAVANE | oral | solution | | |
| hydrochloride | | parenteral | injection | | |
| Trifluoperazine | STELAZINE | | | (2-40 | mg/day) |
| Perphenazine | TRILAFON | oral | solution | 12-64 | mg/day |
| | | | tablets | (16-64 | mg/day) |
| | | parenteral | injection | | |
| Perpehazine and | ETRAFON | oral | tablets | | |
| Amitriptyline | TRIAVIL | | | | |
| hydrochloride | | | | | |
| Thioridazine | MELLARIL | oral | suspension | 150-800 | mg/day |
| | | | solution | (100-300 | mg/day) |
| | | | tablets | | |
| Mesoridazine | | | | (30-400 | mg/day) |
| Molindone | MOBAN | | | 50-225 | mg/day |
| | | | | (15-150 | mg/day) |
| Molindone | MOBAN | oral | solution | | |
| hydrochloride | | | | | |
| Loxapine | LOXITANE | | | 20-250 | mg/day |
| | | | | (60-100 | mg/dav) |
| Loxapine | LOXITANE | oral | solution | | |
| hydrochloride | | parenteral | injection | | |
| Loxapine | LOXITANE | oral | capsules | | |
| succinate | | | | | |
| Pimozide | | | | (1-10 | mg/day) |
| Flupenthixol | | | | | |
| Promazine | SPARINE | | | | |
| Triflupromazine | VESPRIN | | | | |
| Chlorprothixene | TARACTAN | | | | |
| Droperidol | INAPSINE | | | | |
| Acetophenazine | TINDAL | | | | |
| Prochlorperazine | COMPAZINE | | | | |
| Methotrimeprazine | NOZINAN | | | | |
| Pipotiazine | PIPOTRIL | | | | |
| Aripiprazole | | | | | |
| Hoperidone | | | | | |

Examples of tradenames and suppliers of selected neuroleptic drugs are as follows: clozapine (available under the tradename CLOZARIL®, from Mylan, Zenith Goldline, UDL, Novartis); olanzapine (available under the tradename ZYPREX®, from Lilly; ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK); fluphenazine (available under the tradename PROLIXINO, from Apothecon, Copley, Schering, Teva, and American Pharmaceutical Partners, Pasadena); thiothixene (available under the tradename NAVANE®;, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman; perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®)) may be used.

Other availale neuroleptic drugs include promazine (available under the tradename SPARINE®), triflurpromazine (available under the tradename VESPRIN®), chlorprothixene (available under the tradename TARACTAN®)), droperidol (available under the tradename INAPSINE®), acetophenazine (available under the tradename TINDAL®;), prochlorperazine (available under the tradename COMPAZINE®), methotrimeprazine (available under the tradename NOZINAN®), pipotiazine (available under the tradename PIPOTRIL®), ziprasidone, and hoperidone.

Other neuroleptic drugs include the compounds disclosed in the patent application WO03/099786,filed by the same Applicant of the present invention. Among them the compound 7-[4-(4-chloro-benzyloxy)-benzenesulfonyl]-8-methoxy-3-methyl-2,3,4,5-tetrahydro-1H-3-benzazepine and its pharmaceutically acceptable salts are particularly preferred.

In a further particular embodiment of the present invention neuroleptic drugs include risperidone and aripiprazole (from Bristol Myers Squibb Company, see e. g. Stahl SM; Dopamine-system stabilizers, aripiprazole and the next generation of antipsychotics, part 1,“goldilocks”-actons at dopamine receptors; J. Clin. Psychiatry 2001, 62, 11: 841-842).

In a special embodiment of the present invention the neuroleptic drug within the present invention is risperidone (Risperdal®;), its manufacture and pharmacological activity is described in EP 0 196 132. Risperidone acts as an antagonist to neurotransmitters, in particular dopamine, and is used for the treatment of psychoses.

Within the present invention, the neuroleptic risperidone can be administered at a dose of 2-6 mg/day, preferably 4-5 mg. The dose for compounds (I) may range from 0.25 mg/kg to 5 mg/kg, preferably 0.8 mg/kg to 3.0 mg/kg. Preferably, the administration occurs once daily.

Various types of mood stabilisers can be used for the add-on use according to the present invention. Examples of mood stabilisers that are useful in the present invention include, but are not limited to: lithium, valproate, carbamazepine, gabapentin, toplamate, oxcarbazepine, lamotrigine. Lithium in particular may be selected.

The invention is also directed to a novel kit-of-parts that is suitable for use in the treatment of schizophrenic disorders such as schizophrenia, delusional disorders, affective disorders, autism or tic disorders, comprising a first dosage form comprising a neuroleptic agent and a second dosage form comprising the COX-2 inhibitor as defined in the present invention or prodrug thereof, for simultaneous, separate or sequential administration.

According to a further particular embodiment, the dosage form comprising a neuroleptic agent and the second dosage form comprising the COX-2 inhibitor as defined in the present invention are administered simultaneously.

The subject pharmaceutical kit-of-parts may be administered enterally (orally) or parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. In a further particular embodiment of the present invention the administration of a pharmaceutical kit comprising the COX-2 inhibitor as defined in the present invention and a neuroleptic occurs enterally (orally), in form of tablets.

The treatment of schizophrenic disorders with the COX-2 inhibitor as defined in the present invention, alone or in combination with a neuroleptic, may occur in addition to further drug therapies.

Thus, tranquilizers may be used for the treatment of agitation, anxiety or sleep disturbances. Preferably lorazepam is used, which belongs to the class of benzodiazepines.

Experimental Part

The Intermediates and Examples that follow illustrate the invention but do not limit the invention in any way. All temperatures are in OC. Flash column chromatography was carried out using Merck 9385 silica. Solid Phase Extraction (SPE) chromatography was carried out using Varian Mega Bond Elut (Si) cartridges (Anachem) under 15 mmHg vacuum. Thin layer chromatography (TIc) was carried out on silica plates. In addition to those already defined, the following abbreviations are used: Me, methyl; Ac, acyl; DMSO, dimethylsulphoxide; TFA, trifluoroacetic acid; DME, dimethoxyethane; DCM, dichloromethane; NMP. N-methyl pyrrolidone; and MTBE, methyl t-butyl ether.

### EXAMPLE 1

### Preparation of Compounds of Formula (I)

Compounds of formula (I) may be prepared by any method described in WO 02/096885, U.S. application Ser. No. 10/477547 and equivalent patent applications.

Intermediate 1

### 4,4,4-Trifluoro-1-[4-(methylthio)phenyl]butane-1,3-dione

To a solution of ethyl trifluoroacetate (7.95 ml, 1.1 eq) in MTBE (125 ml) was added dropwise 25% sodium methoxide in methanol (16 ml, 1.2 eq). 4-Methylthioacetophenone (Aldrich, 10 g, 0.06 mol) was added portionwise and the mixture. stirred at ambient temperature ovemight. 2N Hydrochloric acid (40 mL) was added cautiously and the organic phase separated, washed with brine and dried (Na₂SO₄) to give an orange solid. The orange solid was recrystallised from hot isopropanol to give the title compound as a yellow crystalline solid (11.25 g, 71%).

MH−261

Intermediate 2

### 2-(Methylthio)-4-[4-(methylthio)phenyl]-6-trifluoromethyl) pyrimidine

To a mixture of 4,4,4-trifluoro-1-[4-(methylthio)phenyl]butane-1,3-dione (5 g) and 2-methyl-2-thiopseudourea sulfate (5.1 g, 0.98 eq) in acetic acid (100 ml) was added sodium acetate (3 g, 2 eq) and heated under reflux for 8 h. The mixture was concentrated in vacuo and water (100 ml) added to give a solid, which was isolated by filtration to give the title compound as a yellow solid (5.8 g, quantitative).

MH+317

Intermediate 3

### 2-(Methylsulfonyl)-4-[4-(methylsulfonyl)phenyl-6-(trifluoromethyl)pyrimidine

To a solution of 2-(methylthio)-4-[4-(methylthio)phenyl]-6-(trifluoromethyl)pyrimidine (5.78 g) in MeOH (500 ml) was added a solution of OXONETM (Aldrich, 56.23 g, 5 eq) in water (200 ml). The mixture was stirred at ambient temperature overnight, concentrated in vacuo and the residue partitioned between water and ethyl acetate (2×100 ml). The combined organic phases were dried and concentrated in vacuo to an off-white solid which was triturated with hot isopropanol to give the title compound as a white solid (5.6 g, 80%).

MH+.381 Tlc SiO₂ Ethyl acetate:cyclohexane (1:1) Rf 0.45

### Example 1.1

### 2-(4-Fluorophenoxy)-4-[4-(methylsulfonyl)phenyl]-6-(tiifluoromethyl)pyrimidine

To a stirred solution of 4-fluorophenol (37 mg, 0.33 mmole) in dry tetrahyrofuran (10 ml) was added, under an atmosphere of nitrogen, sodium hydride (60% dispersion in oil, 13 mg, 0.33 mmole) and the resulting mixture stirred at 20 for 30 min. To the stirred reaction mixture was added 2-(methylsulfonyl)-4-[4-(methylsulfonyl)phenyl]-6-trifluoromethyl)pyrimidine (114 mg, 0.33 mmole) in a single portion, and stirring was continued for 2 h. The solvent was evaporated, and the residue partitioned between dichloromethane and 2N sodium hydroxide. The dried organic phase was evaporated to dryness. The residue was purified on a silica gel SPE cartridge eluting with chloroform to afford the btle compound as a colourless solid (99 mg, 80%).

MH+413.

### Examples 1.2 to 1.10

Examples 1.2 to 1.10,as shown in Table 1 that follows, were prepared in the manner described for Example 1.1

TABLE I

| | |
|---|---|
| | (I) |
| 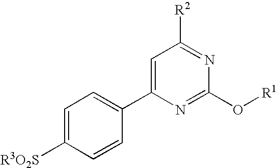 | |

| Ex | R¹ | R² | R³ | MS | |
|---|---|---|---|---|---|
| 1.2 | 3,4-difluorophenyl | CF₃ | CH₃ | MH+ | 431 |
| 1.3 | 4-methoxyphenyl | CF₃ | CH₃ | MH+ | 425 |
| 1.4 | 4-fluorobenzyl | CF₃ | CH₃ | MH+ | 427 |
| 1.5 | 4-bromophenyl | CF₃ | CH₃ | MH+ | 474 |
| 1.6 | 4-methylphenyl | CF₃ | CH₃ | MH+ | 409 |
| 1.7 | 5-chloropyridin-3-yl | CF₃ | CH₃ | MH+ | 431 |
| 1.8 | cyclohexyl | CF₃ | CH₃ | MH+ | 401 |
| 1.9 | cyclopentylmethyl | CF₃ | CH₃ | MH+ | 401 |
| 1.10 | n-butyl | CF₃ | CH₃ | MH+ | 375 |

### Example 1.11

### 2-Butoxy-4-[4-(methylsulfonyl)phenyl]-6-(trifluoromethyl)pyrimidine

Sodium methoxide (6.6 kg of a 30% w/w solution in methanol) was added over at least 30 min to a solution of 4-(methylthio)acetophenone (5.0 kg) and methyl trifluoroacetate (4.25 kg) in tert-butylmethylether (40 L) at 40±3° C. The solution was heated at 40±3° C. for at least 3 h. Acetic acid (55 L) was added, followed by S-methyl 2-thiopseudourea sulfate (5.45 kg) and the mixture concentrated to ca. 45 L. The mixture was heated at about 110° C. for at least a further 8 h (overnight) then acetic acid (20 L) was added before cooling to 50±3° C. A solution of sodium tungstate dihydrate (0.2 kg) in water (2.5 L) was added, followed by hydrogen peroxide (20.7 kg of 30% w/v solution), which was added over at least 3 h, maintaining the temp at ca. 500. The mixture is heated at ca. 50° C. for at least 12 h before cooling to 20±3° C. A solution of sodium sulphite (3.45 kg) in water (28 L) was then added over at least 30 min whilst maintaining the temperature at 20±3° C. The mixture was aged at 20±3° C. for ca. 1 h and 2-(methylsulfonyl)-4-[4-(methylsulfonyl)phenyl]-6-(trifluoromethyl)pyrimidine_{—}collected by filtration, washed with water (3×15 L) and dried at up to 60° in vacuo. Yield, 9.96 kg, 90% of theory.

A suspension of 2-(methylsulfonyl)-4-[4-(methylsulfonyl)phenyl -6-(trifluoromethyl)-pyrimidine (525 g) in n-butanol (5.25 L) was treated with potassium carbonate (210 g) at 20±5° C. The mixture was heated to 50±5° C. overnight until the reaction was complete by HPLC. Acetic acid (1.57 L) was added dropwise, to control any gas evolution, keeping the temperature at 50±5° C. Water (3.67 L) was then added over 30 min keeping the temperature at 50±5° C. to allow full crystallisation to occur. The slurry was then cooled to 20-25° C. and aged for at least 1 hour. The resulting product was then filtered under vacuum and washed with a mixture of n-butanol (787 mL), acetic acid (236 mL), and water (551 mL) followed by water (2×1.57 L). The product was then dried at up to ca 50° C. under vacuum to yield the title compound. Yield, 457 g, 88.4% of theory. The title compound was found to be identical to that of Example 10.

¹ H NMR (CDCl₃) δ: 8.33(2H, d, para-di-substituted CH); 8.11(2H, d, para-di-substituted CH); 7.70(1H, s, aromatic CH); 4.54(2H, t, butyl CH₂); 3.12(3H, s, sulphone CH₃); 1.88(2H, m, butyl CH₂); 1.55(2H, m, butyl CH₂); 1.01 (3H, t, butyl CH₃).

### EXAMPLE 2

### Preparation of Compounds of Formula (II)

Compounds of formula (II) may be prepared by any method described in WO 99/12930, U.S. Pat. No 6,451,794, US-A-2003-0040517 and US-A-2003-0008872 and equivalent patent applications.

### Example 2.1

### 6-Difluoromethoxy-2-(4-fluoro-phenyl)-3-(4-methanesulfonyl-phenyl)-pyrazolo[1,5-b]pyridazine

### (i) 6-Methoxy-2-(4-fluoro-phenyl-pyrazolo[1,5-b]pyridazine-3-carboxylic acid methyl ester

1,8-Diazabicyclo[5.4.0]undec-7-ene (3.39 mL) was added to a mixture of 3-4-fluorophenyl)-prop-2-ynoic acid methyl ester (3.36 g) and 1-amino-3-methoxy-pyridazin-1-ium mesitylene sulphonate¹ (6.1419 g) in acetonitrile (125 mL) and the mixture was stirred at ambient temperature for 48 hours. During the first 2 hours a stream of air was passed through the reaction. The mixture was concentrated in vacuo, dissolved in ethyl acetate (150 mL), washed with water (3×25 mL), dried (MgSO₄), filtered and evaporated in vacuo to give the title compound as a brown solid (4.77 g).
Ref:¹ T. Tsuchiya, J. Kurita and K. Takayama, Chem. Pharm. Bull. 28(9) 2676-2681 (1980).

¹H NMR (CDCl₃): 8.4 (d, 1H, J=10Hz) 7.85-7.90 (m, 2H) 7.1-7.2 (m, 2H) 6.9-7.0 (d, 1H, J=10 Hz) 4.1 (s, 3H) 3.9 (s, 3H) MH⁺ 302

### (ii) 6-Methoxy-2-(4-fluoro-phenyl-pyrazolo[1,5-b]pyridazine-3-carboxylic acid

A mixture of 6-methoxy-2-(4-fluoro-phenyl-pyrazolo[1,5-b]pyridazine-3-carboxylic acid methyl ester (4.469 g), 2N sodium hydroxide (50 mL) and methanol (90 mL) was heated at reflux for 2 hours. The cooled solution was added to 2N hydrochloric acid (200 mL) and the title compound was isolated by filtration as a beige solid (3.639 g).

¹H NMR (DMSO-d₆): 12.8 (br. s, 1H) 8.4 (d, 1H, J=10 Hz) 7.8-7.9 (m, 2H) 7.21-7.32 (m, 2H) 7.15-7.2 (d, 1H, J=10 Hz) 4.0 (s, 3H) MH⁺ 288

### (iii) 2-(4-Fluoro-phenyl)-3-(4-methanesulfonyl-phenyl)-6-methoxy-pyrazolo[1,5-b]-pyridazine

A mixture of 6-methoxy-2-(4-fluoro-phenyl-pyrazolo[1,5-b]pyridazine-3-carboxylic acid (869 mg) and sodium bicarbonate (756 mg) in dimethylformamide (10 mL) was treated with N-bromosuccinimide (587 mg) and stirred at ambient temperature for 1 hour, then added to water (50 mL) and extracted with ethyl acetate (3×50 mL), dried (MgSO₄), and evaporated in vacuo. The resulting brown solid (1.612 g) was dissolved in 1,2 dimethoxyethane (20 mL). 2N Aqueous sodium carbonate solution (10 mL) was added together with 4-(methanesulphonyl)phenyl boronic acid (660 mg) and tetrakis(triphenylphosphine)palladium (0) (100 mg) and the mixture was heated at reflux for 20 hours. The reaction was poured into water (50 mL), extracted with dichloromethane (3×100 mL). The combined organic extracts were dried (MgSO₄) and evaporated in vacuo to give a brown solid (1.116 g) which was purified by flash column chromatography on silica, eluting with cyclohexane/ethyl acetate (4:1 then 2:1), to give the title compound as a yellow solid (390 mg).

Tlc, SiO₂, R_{f} 0.3 (1:1 cyclohexane/ethyl acetate), detection UV MH⁺ 398

### (iv) 2-(4-Fluoro-phenyl)-3-(4-methanesulfonyl-phenyl)-pyrazolo1,5-b]pyridazin-6-ol

A mixture of 2-(4-fluoro-phenyl)-3-(4-methanesulfonyl-phenyl)-6-methoxy-pyrazolo[1,5-b]pyridazine (321 mg) and pyridine hydrochloride (1.4 g) was heated to and at 200° C. in a sealed vessel (Reactivial™) for 3 hours. The cooled reaction was poured into water (20 mL), and extracted with ethyl acetate (3×30 mL). The combined organic extracts dried (MgSO₄), filtered and evaporated in vacuo to give a solid which was triturated with diethyl ether to give the title compound as a beige solid (119 mg).

Tlc, SiO₂, Rf 0.07 (1:2 cyclohexane/ethyl acetate), detection UV. MH⁺ 384

### (v) 6-Difluoromethoxy-2-(4-fluoro-phenyl)-3-(4-methanesulfonyl-phenyl)-pyrazolo[1,5-b]pyridazine

A solution of 2-(4-fluoro-phenyl)-3-(4-methanesulfonyl-phenyl)-pyrazolo[1,5-b]pyridazin-6-ol (0.2 g) in anhydrous dimethyl formamide (5 mL) was treated with sodium hydride (0.0469, 60% dispersion in mineral oil), after effervescence ceased a stream of bromodifluoromethane gas was passed through the mixture at ambient temperature for 30 minutes. The reaction mixture was then poured into water (50 mL) and extracted with ethyl acetate (50 mL), the organic extract was washed with water (3×50 mL), dried and concentrated in vacuo. The residue was purified by chromatography to give the title compound as a white solid (0.17 g).

MH⁺=434 ¹HNMR(CDCl₃): δ88.05-8.0(d, J=10 HZ, 2H)8.0-7.95(d, J=10 HZ, 1H)7.6-7.5(m,4H)7.8-7.2(t, J=70 HZ, 1H)7.1-7.05(t, J=11 HZ, 2H)6.9-6.85(d, J=10 HZ, 1H)3.15(s,3H) Tlc, SiO₂, Rf 0.35(ethyl acetate/cyclohexane(1/1))

### Example 2.2

### 3-(4-Methanesulfonyl-phenyl)-2-(4-methoxy-phenyl)-pyrazolo[1,5-b]pyridazine

### (i) 2-(4-Methoxy-phenyl)-pyrazolo[1,5-b]pyridazine-3-carboxylic acid methyl ester

Diazabicyclo[5.4.0]undec-7-ene (22.76 mL, 2 eq) was added dropwise to a solution of methyl 3-(4-methoxy-phenyl)-prop-2-ynoic acid¹ (14.46 g, 76 mM) and 1-amino pyridazinium iodide² (2 eq) in acetonitrile under nitrogen and stirred for 6 h. Purification by chromatography on silica gel eluting with toluene, then toluene:ethyl acetate (9:1) gave the title compound (2.76 g) as a brown solid.
Ref:¹ J. Morris and D. G. Wishka, Synthesis (1994), (1), 43-6

Ref:² Kobayashi et al Chem. Pharm. Bull. (1971), 19 (10), 2106-15

MH⁺ 284 1H NMR (CDCl₃) δ 3.87 (3H, s) 3.9 (3H, s) 7.0 (2H, d, J=9 Hz) 7.25 (1H, dd, J=9 & 4 Hz) 7.90 (2H, d, J=9 Hz) 8.45 (1H, dd, J=4 & 2 Hz) 8.55 (1H, dd, J=9 & 2 Hz)

### (ii) 3-(4-Methanesulfonyl-phenyl)-2-(4-methoxy-phenyl)-pyrazolo[1,5-b]pyridazine

A mixture of 2-(4-methoxy-phenyl)-pyrazolo[1,5-b]pyridazine-3-arboxylic acid methyl ester (2.76 g) and aq. sodium hydroxide (2N, 30 mL) in ethanol (30 mL) was refluxed under nitrogen for 2 h. The cooled mixture was acidified with hydrochloric acid (2N) and the resulting white solid (2.53 g) isolated by filtration. This solid was dissolved in DMF and sodium bicarbonate (2.67 g, 3.3 eq) added, followed by N-bromosuccinimide (1.88 g, 1.1 eq) portionwise. After stirring for 1 h under nitrogen, water was added and extracted into ethyl acetate (2×25 mL). The dried organic phase was concentrated and the residue taken up in DME (60 mL). Aqueous sodium carbonate (2N, 15 mL) was added, followed by 4-methanesulfonyl-phenylbbronic acid (3.12 g) and tetrakis(triphenylphosphine)palladium(0) (250 mg). The mixture was heated at reflux under nitrogen for 18 h, cooled, poured into water and extracted into ethyl acetate (2×25 mL). The combined organic phases were dried and concentrated onto silica gel. Chromatography on silica gel eluting with toluene:ethyl acetate (8:1) gave, on concentration, the title compound (3.58 g) as a cream solid.

MH⁺ 380 1H NMR (DMSO) δ 3.25 (3H, s) 3.75 (3H, s) 6.95 (2H, d, J=8.5 Hz) 7.25 (1H, dd, J=9 & 5 Hz) 7.45 (2H, d, J=8.5 Hz) 7.60 (2H, d, J=8 Hz) 7.9 (2H, d, J=8.5 Hz) 8.15 (1H, dd, J=9&2 Hz) 8.49 (1H, dd, J=5&2 Hz)

### Example 2.3

### 2-(4-Ethoxy-phenyl)-3-(4-methanesulfonyl-phenyl)-pyrazolo[1,5-b]pyridazine

### (i) 4-[3-(4-Methanesulfonyl-phenyl)-pyrazolo[1,5-b]pyridazin-2-yl]-phenol

Boron tribromide (1M solution in CH₂Cl₂, 2.1 eq) was added to 3-(4-methanesulfonyl-phenyl)-2-(4-methoxy-phenyl)-pyrazolo[1,5-b]pyridazine (3.58 g) in CH₂Cl₂ at −70°. The mixture was stirred for 10 min then warmed to 0° and stirred at 0° overnight. The reaction mixture was made alkaline with potassium carbonate then acidified with hydrochloric acid (2M), poured into water and extracted into CH₂Cl₂. The organic phase was dried, filtered and concentrated to give the title compound (1.87 g) as a yellow solid.

MH⁺ 366 1H NMR (DMSO) δ 3.30 (3H, s) 6.80 (2H, d, J=8.5 Hz) 7.30 (1H, dd, J=9 & 5 Hz) 7.35 (2H, d, J=8.5 Hz) 7.60 (2H, d, J=8 Hz) 8.0 (2H, d, J=8.5 Hz) 8.20 (1H, dd, J=9& 2 Hz) 8.55 (1H, dd, J=5& 2 Hz) 9.75 (1H, s)

### (ii) 2-(4-Ethoxy-phenyl)-3-(4-methanesulfonyl-phenyl)-pyrazolo[1,5-b]pyridazine

4-[3-(4-Methanesulfonyl-phenyl)-pyrazolo[1,5-b]pyridazin-2-yl]-phenol (663 mg, 1.82) iodoethane (eq) and potassium carbonate (2 eq) in acetonitrile (30 mL) were heated at reflux under nitrogen for 18 h. The cooled reaction mixture was partitioned between water (30 mL) and ethyl acetate (30 mL). The organic phase was collected, dried and purified by chromatography to give the title compound (547 mg) as a cream foam.

MH⁺ 394 1H NMR (DMSO) δ 1.45 (3H, t, J=7 Hz) 3.10 (3H, s) 4.1 (2H, q, J=7 Hz) 6.87 (2H, d, J=9 Hz) 7.08 (1H, dd, J=9 & 5 Hz) 7.55 (4H, t, J=9 Hz) 7.92(1H, dd, J=9 &2Hz) 7.95 (2H, d, J=9 Hz) 8.20 (1H, dd, J=9& 2 Hz) 8.32 (1H, dd, J=!5& 2 Hz)

### Example 2.4

### 2-(4-Fluoro-phenyl)-6-methanesulfonyl-3-(4-methanesulfonyl-phenyl)-pyrazolo[1,5-b]pyridazine

### (i) 2-(4-Fluoro-phenyl)-6-methylsulfanyl-pyrazolo[1,5-b]pyridazine-3-carboxylic acid methyl ester

Solid t-butoxycarbonyl-O-mesitylenesulfonylhydcroxylamine¹ (7.8 g) was added portionwise with stirring to TFA (25 mL) over 10 min then stirred for a further 20 minutes. The solution was poured onto ice (˜200 mL) and left until the ice melted. The resulting white solid was filtered off, washed with water, and dissolved in DME (100 mL). The solution was dried over 4 A mol. sieves for 1.5 hours, filtered then added to a solution of 3-methylthio-pyridazine² (2.6 g) in dichloromethane (35 mL) and the reaction stirred at room temperature for 20 h. The intermediate salt was isolated by filtration as light brown crystals (3.87 g), suspended in acetonitrile (100 mL) and methyl 3-(4-fluoro-phenyl)-prop-2-ynoic acid (2.02 g) added. 1,8Diazabicyclo[5.4.0]undec-7-ene (2.1 mL) was added dropwise and the reaction was stirred at room temperature for 20 hours. The resulting crystalline precipitate was filtered off, washed and dried (770 mg). Concentration of the filtrate gave a second crop (430 mg). The residues were partioned between water and ethyl acetate (100 mL each) and the aqueous layer was extracted with ethyl acetate (20 mL). The combined organics were washed with water, brine and dried. Removal of solvent gave a brown oil which was purified by flash chromatography on silica (300 g) eluting with cyclohexane/ethyl acetate (3:1) to give a further quantity of product (247 mg). The three crops were combined to give the title compound (1.45 g) as a light brown solid.
Ref:¹ K Novitskii et al, Khim Geterotskil Soedin, 1970 2, 57-62

Ref:² Barlin G. B., Brown, W. V., J Chem Soc (1968), (12), 1435-45

MH⁺ 318 1H NMR (CDCl₃) δ 2.70 (3H, s ), 3.88 (3H, s) 7.08-7.18 (3H, m) 7.84 (2H, m) 8.31 (1H, d, J=10 Hz)

### (ii) 2-(4-Fluoro-phenyl)-3-(4-methanesulfonyl-phenyl)-6-methylsulfanyl-pyrazolo[1,5-b]pyridazine

A mixture of the 2-(4-fluoro-phenyl)-6-(methylthio)-pyrazolo[1,5-b]pyridazine-3-carboxylic acid methyl ester (1.45 g) potassium carbonate (690 mg) in methanol (40 mL) and water (14 mL) was stirred and heated under reflux for 20 hours under nitrogen. The solvents were removed and the resulting solid partioned between ethyl acetate (50 mL) and water (250 mL). The aqueous layer was acidified to pH1 (2MHCI) and a solid was filtered off (1.0 g, MH⁺ 304). A mixture of the solid (1.0 g), sodium bicarbonate (557 mg) and NBS (594 mg) were stirred at room temperature for 4 hours. The reaction was poured into water (150 mL) and extracted with ethyl acetate (3×50 mL). The combined extracts were washed with water (50 mL), brine (20 mL), dried and concentrated. The resulting solid (1.015 g, MH⁺ 338, 340), 4-(methanesulphonyl)phenyl boronic acid (902 mg), sodium carbonate (740 mg) and tetrakis(triphenylphosphine)palladium(0) (175 mg) were stirred and heated under nitrogen at reflux in DME (30 mLs) and water (15 mL) for 48 hours. The reaction was poured into water and extracted with ethyl acetate (3×50 mL). The combined extracts were dried and the solvent removed to give a brown solid. This was purified on silica (300 g) eluting with cyclohexane, ethyl acetate (1:1) to give the title compound (0.713 g) as a yellow solid.

MH⁺ 414 1H NMR δ (DMSO) 2.65 (3H, s) 3.28 (3H, s) 7.20-7.30 (3H, m) 7.55 (2H, m) 7.62 (4H, d, J=8.5 Hz) 7.95-8.05 (3H, m)

### (iii) 2-(4-Fluoro-phenyl)-6-methanesulfonyl-3-(4-methanesulfonyl-phenyl)-pyrazolo[1,5-b]pyridazine

A suspension of 2-(4-fluoro-phenyl)-3-(4-methanesulfonyl-phenyl)-6-(methylthio)-pyrazolo[1,5-b]pyridazine (60 mg 0.145) in MeOH (5 mL) and water (2 mL) was stirred with oxone (196 mg 0.32) for 20 hours. The resulting solution was poured into water (50 mL) and extracted with chloroform (3×20 mL). The combined extracts were dried and the solvent removed. Crystallisation of the residue from methanol gave the title compound (60 mg) as a white solid.

MH⁺ 446 1H NMR (DMSO-d6) δ 3.34 (3H, s) 3.53 (3H, s) 7.33 (2H, t, J=9 Hz) 7.62 (2H, m) 7.68 (1H, d, J=8.5 Hz) 8.04 (1H, d, J=10 Hz) 8.52 (1H, d, J=9 Hz) TLC SiO₂ Hexane:Ethyl acetate (1:1) Rf 0.24 UV

### Example 2.5

### 2-(4-Difluoromethoxy-phenyl)-3-(4-methanesulfonyl-phenyl)-pyrazolo[1,5-b]pyridazine

Sodium hydride (48 mg, 60% disp. in oil, 1.2 mmol) was added to a solution of 4-[3-(4-methanesulfonyl-phenyl)-pyrazolo[1,5-b]pyridazin-2-yl]-phenol (200 mg, 0.55 mmol) in anhydrous dimethylformamide (5 mL). Bromodifluoromethane gas was gently bubbled through the solution for 20 min, then diluted with CH₂Cl₂ (30 mL). Aqueous workup followed by chromatography on silica gel with CH₂Cl₂:ethyl acetate (3:1) as eluant then chromatography with CH₂Cl₂:ethyl acetate (10:1) as eluant gave the title compound (63 mg, 28%) as a white solid.

MH⁺ 416 NMR (CDCl₃) δ 8.38 (1H, dd, J=4 Hz), 8.01 (2H, d, J=8.5 Hz), 7.94 (1H, dd, J=9 &2 Hz), 7.65 (2H, d, J 8.5 Hz) 7.57 (2H, d, J=8 Hz), 7.10 (3H, m), 6.87-6.27 (1H, t, J=7.4 Hz) 3.15 (3H, s)

### Example 2.6

### 4-[2-(4-Ethoxy-phenyl)-pyrazolo[1,5-b]pyridazin-3-yl]-benzenesulfonamide

### (i) 2-(4-Ethoxy-phenyl)-pyrazolo[1,5-b]pyridazine-3-carboxylic acid methyl ester

Diazabicyclo[5.4.0]undec-7-ene (1.47 mL, 2 eq) was added dropwise to a solution of methyl 3-(4-ethoxy-phenyl)-prop-2-ynoic acid (1.0 g) and 1-amino pyridazinium iodide² (2.19 g) in acetonitrile (10 mL) under nitrogen and stirred for 5 h. Concentration and aqueous workup gave the title compound (1.2 g) as a sticky brown solid.

MH⁺ 298

### (ii) 2-(4-Ethoxy-phenyl)-pyrazolo[1,5-b]pyridazine-3-carboxylic acid

A mixture of 2-(4-ethoxy-phenyl)-pyrazolo[1,5-b]pyridazine-3-carboxylic acid methyl ester (1.2 g), ethanol (10 mL) and 2N sodium hydroxide (10 mL) was heated to 80° for 1.5 h. The mixture was allowed to cool and acidified to pH 1 with 2N hydrochloric acid. The title comoound was isolated by filtration as a brown solid (716 mg, 63%).

MH⁺ 284

### (iii) 2-(4-Ethoxy-phenyl)-3-iodo-pyrazolo[1,5-b]pyridazine

A mixture of 2-(4-ethoxy-phenyl)-pyrazolo[1,5-b]pyridazine-3-carboxylic acid (710 mg), N-iodosuccinimide (678 mg) and sodium bicarbonate (717 mg) in DMF (8 mL) was stirred for 4 h. A further quantity of N-iodosuccinimide (100 mg) was added and stirring continued for 2 h. Aqueous workup gave a dark brown solid which was purified by SPE with dichloromethane as eluant. This gave the title compound as an orange-brown solid (429 mg, 47%).

MH⁺ 366

### (iv) 4-[2-(4-Ethoxy-phenyl)-pyrazolo[1,5-b]pyridazin-3-yl]-benzenesulfonamide

A mixture of 4-iodobenzenesulphonamide (0.311 g), dipinacoldiborane¹ (0.279 g), potassium acetate (486 mg) and [1,1′-bis(diphenylphosphino)-ferrocene]palladium(II) chloride complex with dichloromethane (1:1) (0.45 g) in dimethylformamide (8 mL) was heated under nitrogen at 80° for 2 h. The cooled reaction mixture was concentrated in vacuo and the residue suspended in 1,2 dimethoxyethane (10 mL), 2-(4-ethoxy-phenyl)-3-iodo-pyrazolo[1,5-b]pyridazine (0.4 g) was added together with 2N sodium carbonate (4 mL) and tetrakis(triphenylphosphine)palladium (0) (20 mg) and the mixture heated at reflux under nitrogen for 18 hours. The cooled reaction mixture was poured into water (60 mL) and the suspension extracted with ethyl acetate (3×60 mL). The organic extracts were combined, dried (Na₂SO₄) and concentrated. The residue was purified by chromatography eluting with dichloromethane/ethyl acetate (3:1) to give the title compound as a yellow solid (0.116 g, 27%).
Ref: ¹ R. Miyaura et al J. Org. Chem., 1995, 60, 7508-7510.

MH⁺ 395 NMR (CDCL₃) δ 8.32 (1H, dd, J=4 & 2 Hz), 7.97 (2H, d, J=8 Hz), 7.89 (1H, dd, J=9 & 2 Hz), 7.54 (4H, m), 7.04 (1H, dd, J=9 & 4 Hz), 6.88 (2H, d, J=9 Hz), 1.43 (3H,

### Example 2.7

### 6-Difluoromethoxy-2-(3-fluoro-phenyl)-3-(4-methanesulfonyl-phenyl)-pyrazolo[1,5-b]pyridazine

### (i) 1-(2,2-Dibromo-vinyl)-3-fluoro-benzene

To a stirred cooled (ice/salt, 0°) solution of carbon tetrabromide (48.82 g) in anhydrous CH₂Cl₂ (200 mL) was added portionwise over 3 minutes, triphenylphosphine (77.1 g), maintaining the temperature below 10°. The resulting orange suspension was stirred at 0° for 1 hour before adding to it, 3-fluorobenzaldehyde (7.8 mL). After the addition was complete, the suspension was stirred at 0° for 1 hour then quenched by the addition of water.(75 mL). The organic phase was separated and washed with brine (75 mL), dried (Na₂SO₄) and evaporated to dryness. The residual gum was poured into cyclohexane (1 L) and stirred for 30 minutes. The organic phase was decanted and the residue taken up into CH₂Cl₂ and poured into cyclohexane (1 L). This procedure was repeated twice more and the combined organic phases concentrated to ˜100 mL and passed through silica gel. The filtrate was concentrated to give the title compound as a mobile yellow oil (24 g, 100%).

MH⁺ 280, MH⁻ 279 NMR (CDCl₃) δ 7.05 (1H, tm, J=9 Hz) 7.3 (3H, m) 7.45 (1H, s)

### (ii) (3-Fluoro-phenyl)-propynoic acid methyl ester

To a stirred solution of 1-(2,2-dibromo-vinyl)-3-fluoro-benzene (23.8 g) in anhydrous THF (350 mL) cooled to −78° was added dropwise over 30 minutes, n-butyllithium (2.2 eq, 1.6M in hexanes). The mixture was stirred for a further 30 minutes at −78° before methyl chloroformate (11.6 g, 9.5 mL) was added and the resultant mixture allowed to warm to 0° for 1 hour before being diluted with 1:1 saturated aqueous sodium bicarbonate:ammonium chloride (100 mL) and extracted into ether (2×100 mL). The combined organic extract was washed with brine (25 mL), dried (Na₂SO₄) and evaporated to dryness to give the title compound as a brown oil (16.7 g, 100%).

MH⁻ 173 NMR (CDCl₃) δ 7.4-7.1 (4H, m) 3.85 (3H, s, CO₂Me)

### (iii) 2-(3-Fluoro-phenyl)-6-methoxy-pyrazolo[1,5-b]pyridazine-3-carboxylic acid methyl ester

1,8-Diazabicyclo[5.4.0]undec-7-ene (5 mL) was added to a stirred, chilled, mixture of (3-fluoro-phenyl)-propynoic acid methyl ester (2.67 g) and 1-amino-3-methoxy-pyridazin-1-ium mesitylene sulphonate (4.89 g) in acetonitrile (80 mL) and the mixture was stirred at 0° for 1 hour then at ambient temperature for 18 hours. The mixture was concentrated in vacuo, and partitioned between ethyl acetate (150 mL) and water (150 mL). The aqueous phase was separated and further extracted with ethyl acetate (2×100 mL). The combined organic extracts were washed with water (2×50 mL), brine (25 mL), dried (MgSO₄), filtered and evaporated in vacuo to give a solid which was triturated with anhydrous ether petroleum ether (1:0.5) to give the title compound as a brown solid (2.4 g, 53%).

MH⁺ 302 1H NMR (CDCl₃) δ 12.8 (1H, br s); 8.4 (1H, d, J 10 Hz) 7.7-7.6 (2H, m) 7.42 (1H, q, J 8 Hz) 7.15 (1H, td, J 8 & 3 Hz) 6.95 (1H, d, J 10 Hz) 4.1 (3H, s) 3.88 (3H, s)

### (iv) 2-(3-Fluoro-phenyl)-6-methoxy-pyrazolo[1,5-b]pyridazine-3-carboxylic acid

2N sodium hydroxide (50 mL) was added to a solution of 2-(3-fluoro-phenyl)-6-methoxy-pyrazolo[1,5-b]pyridazine-3-carboxylic acid methyl ester (2.3 g) in absolute ethanol (50 mL) and the resulting mixture heated to reflux for three hours. The cooled reaction mixture was poured slowly into a stirred solution of 2N hydrochloric acid (300 mL). The resulting suspension was stirred at ambient temperature for 1 hour then filtered and the filter cake washed with water and dried in vacuo at 60° to give the title compound as an off-white solid (2.0 g, 91%).

MH⁺ 288 1H NMR (DMSO) δ 8.45 (1H, d, J 10 Hz); 7.67 (2H, m); 7.5 (1H, q, J 7 Hz); 7.3 (1H, 7 & 2 Hz); 7.21 (1H, d, J 10 Hz); 4.0 (3H, s)

### (v) 3-Bromo-2-(3-fluoro-phenyl)-6-methoxy-pyrazolo[1,5-b]pyridazine

To a stirred solution of 2-(3-fluoro-phenyl)-6-methoxy-pyrazolo[1,5-b]pyridazine-3-carboxylic acid (2.0 g) in anhydrous DMF (20 mL) was added n-bromosuccinimide (1.78 g) and the resulting solution stirred at ambient temperature for 3 hours. The reaction mixture was diluted with ethyl acetate (800 mL) and washed sequentially with water (10×100 mL) and sat. brine (25 mL), dried (Na₂SO₄), and concentrated to give the title compound as a buff solid (2.1 g, 93%).

MH⁺ 323, MH⁻ 321 1H NMR (CDCl₃) 7.9 (2H, m) 7.8 (1H, d, J 10 Hz); 7.45 (1H, m); 7.1 91H, td, J 8 & 2 Hz); 6.78 (1H, d, J 10 Hz); 4.1 (3H, s)

### (vi) 6-Difluoromethoxy-2-(3-fluoro-phenyl)-pyrazolo[1,5-b]pyridazine

Portions of 3-bromo-2-(3-fluoro-phenyl)-6-methoxy-pyrazolo[1,5-b]pyridazine (400 mg, 2.1 g total) were placed in individual Reactivials equipped with a magnetic stirrer bar. Pyridine hydrochloride (10 eq) was added to each vial, the vials sealed, and heated to 200° for 3 hours. The vials were allowed to cool to −140° before opening and the contents poured into ice/water. The resulting mixture was extracted into ethyl acetate (3×100 mL) and the combined organic extracts washed with water (7×75 mL), dried (Na₂SO₄) and evaporated to give the des-bromo phenol as a brown solid (1.0 g, MH⁺ 230).

This solid was dissolved in anhydrous DMF (10 mL) and sodium hydride (60% dispersion in mineral oil, 200 mg) added portionwise. After stirring for 20 minutes at ambient temperature the solution was transferred to a small cooled autoclave and bromodifluoromethane (5 mL, xs, condensed at −30°) added. The autoclave was then sealed, allowed to warm to ambient temperature and stirred for 36 hours. The resulting solution was diluted with ethyl acetate (200 mL), washed with water (10×20 mL), dried (Na₂SO₄), concentrated and the residual gum purified by flash column chromatography with cyclohexane:ethyl acetate (4:1) as eluant. This gave the title compound as a solid (652 mg, 60%).

MH⁺ 280 MH³¹ 278 NMR (DMSO) δ 8.42(1H, d, J=10 Hz) 7.85 (1H, d, J 8 Hz) 7.78 (1H, t, J 70 Hz) 7.55 q, J 8 Hz) 7.38 (1H, s) 7.25 (1H, m) 7.17 (1H, d, J 10 Hz)

### (vii) 3-Bromo-6-difluoromethoxy-2-(3-fluoro-phenyl)-pyrazolo[1,5-b]pyridazine

N-bromo succinimide (195 mg) was added to a solution of 6-difluoromethoxy-2-(3-fluoro-phenyl)-pyrazolo[1,5-b]pyridazine (251 mg) and sodium bicarbonate (185 mg) in anhydrous DMF (10 mL) and stirred for 18 h. The reaction mixture was diluted with ethyl acetate (300 mL) and washed with water (10×20 mL), brine (20 mL), dried (Na₂SO₄) and concentrated to give the title compound as a solid (293 mg, 91%).

MH⁺ 359, MH⁻ 356/357 NMR (DMSO) δ 8.36 (1H, d, J 10 Hz) 7.88 (1H, d, J δ 8 Hz) 7.78 (1H, t, J 70 Hz, OCHF₂) 7.77 (1H, dm, J 10 Hz) 7.62 (1H, dt, J 8 & 6 Hz) 7.38 (1H, dt, J 9 & 2 Hz) 7.3 (1H, d, J 10 Hz)

### (viii) 6-Difluoromethoxy-2-(3-fluoro-phenyl)-3-(4-methanesulfonyl-phenyl)-pyrazolo[1,5-b]pyridazine

To a stirred solution of 3-bromo-6-difluoromethoxy-2-(3-fluoro-phenyl)-pyrazolo[1,5-b]pyridazine (286 mg) in DMF (20 mL) was added 2N aq sodium carbonate (10 mL). To this mixture was added 4-methanesulfonyl-phenylboronic acid (180 mg) and tetrakis triphenylphosphine palladium (0) (34 mg). The resulting mixture was stirred and heated to reflux for 18 hours. The cooled reaction mixture was diluted with ethyl acetate (300 mL) and the organic solution washed with water (1×30 mL) and brine (30 mL), dried (Na₂SO₄) and evaporated to give a gum which was purified by flash column chromatography with chloroform:ethyl acetate (50:1 to 5:1) as eluant. Combination of appropriate fractions and concentration gave the title compound as an off-white solid (132 mg, 37%).

MH⁺ 434 1H NMR (CDCl₃) δ 8.02 (1H, d, J 9 Hz); 7.95 (2H, d, J 10 Hz); 7.58 (1H, d, 9 Hz); 7.52 (1H, t, J 70 Hz); 7.32 (3H, m); 7.08 (1H, m); 6.9 (1H, d, J 9 Hz); 3.15 (3H, s)

### EXAMPLE 3

### Preparation of Compounds of Formula (III)

Compounds of formula (III) may be prepared by any method described in WO 2004/024691 and equivalent patent applications.

### Example 3.1

### N-cyclohexyl-4-(trifluoromethyl)-6-[4-(methylsulfonyl)phenyl]pyridine-2-amine

### (i) 2-[4-(methylthio)phenyl]-4-(trifluoromethyl)-pyridine

To a mixture of 2-chloro-4-(trifluoromethyl)pyridine (19.9 g, 0.11 mol), 4-(methylthio)phenylboronic acid (21.9 g, 0.13 mol), 1 M aqueous sodium carbonate (180 mL) and 1,2-dimethoxyethane (270 mL) under an atmosphere of nitrogen was added palladium tetrakistriphenylphosphine (3.78 g, 3.3 mmol) and the reaction heated at 100° C. for 14 hours. After cooling and concentration in vacuo, the residue was partitioned between ethyl acetate (350 mL) and water (400 mL) and separated. The aqueous layer was further extracted with ethyl acetate (2×150 mL) and the combined organic layers were dried over sodium sulfate and concentrated in vacuo. Filtration through a pad of silica gel (200 g) eluting with a gradient of ethyl acetate in cyclohexane gave the title compound (29.4 g) LC retention time 3.62 mins, MS m/z 269 (MH^{+).}

### (ii) 2-[4-(methylsulfonyl)phenyl]-4-(trifluoromethyl)-pyridine

To a stirred suspension of intermediate (i) (29.4 g, 0.11 mol) in methanol (400 mL) at 0° C. was added portionwise a suspension of Oxone™ (134 g) in water (200 mL). The reaction was warmed to room temperature and stirred for 14 hours. The methanol was removed in vacuo and the residue diluted with saturated aqueous sodium bicarbonate (2 L) and extracted with ethyl acetate (3×1 L). The combined organic layers were dried over sodium sulfate and concentrated in vacuo to give the title compound (32 g, 0.106 mol) LC retention time 2.90, MS m/z 302 (MH⁺)

### (iii) 2-Chloro-4-(trifluoromethyl)-6-[4-(methylsulfonyl)phenyl]pyridine

To a solution of intermediate (ii) (32 g, 0.106 mol) in dichloromethane (400 mL) at reflux was added 3-chloroperbenzoic acid (41.7 g of 57 to 86% grade material) portionwise over 15 minutes. After stirring for 14 hours at reflux, the reaction was cooled, diluted with dichloromethane (2 L) and washed sequentially with saturated aqueous sodium bicarbonate solution, saturated aqueous sodium sulfite solubon containing tetra-n-butylammonium sulfate (4 mL) and water, dried over sodium sulfate and concentrated in vacuo to give 2-[4-(methylsulfonyl)phenyl]-4-(trifluoromethyl)-pyridine-N-oxide (37.2 g, containing traces of a tetra-n-butylammonium salt) LC retention time 2.34, MS m/z 318 (MH⁺). A mixture of this crude material and phosphorus oxychloride (110 mL) was heated at 110° C. for 4 hours. After cooling, the majority of the phosphorus oxychloride was removed in vacuo and the residue neutralised with saturated aqueous sodium bicarbonate solution (300 mL), with cooling. The mixture was extracted with chloroform and the combined organic extracts dried over sodium sulfate and concentrated in vacuo. The residue was recrystallised from 2-propanol to give the title compound (22.0 g) LC retention time 3.23 min, MS m/z 336/338 (MH⁺).

### (iv) N-cyclohexyl-4-(trifluoromethyl)-6-[4-(methylsuffonyl)phenyl]pyridine-2-amine

A stirred mixture of intermediate (iii) (6 g, 17.8 mmol) and cyclohexylamine (175 mL) was heated at 110° C., for 14 hours. After cooling, the reaction was diluted with water (1 L), acidified with 2N HCI (750 mL) and filtered to give the title compound (6.48 g) LC retention time 3.81 mins MS m/z 399 (MH⁺); ¹H-NMR (CDCl₃) δ 1.22-1.86 (8H, m), 2.60-2.16 (2H, m), 3.09 (3H, s), 3.67-3.78 (1H, m), 4.84 (1H, d, J=7 Hz), 6.57 (1H, s), 7.19 (1H, s), 8.03 (2H, d, J=9 Hz), 8.17 (2H, d, J=9 Hz).

### Example 3.2

### 2-[4-(methylsulfonyl)phenyl]-6-[(2-pyridinylmethyl)oxy]-4-(trifuoromethyl)pyridine

### (i) 4-(Trifluoromethyl)-6-[4-(methylthio)phenyl]-2-pyridone

To a stirred solution of diisopropylamine (11.5 mL, 81.8 mmol) in THF (75 mL) at 0° C. was added n-butyllithium (51.1 mL of a 1.6M solution in hexanes, 81.8 mmol). After stirring for 15 minutes, a solution of 4,4,4-trifluoro-3-methyl-2-butenoic acid (6.0 g, 38.9 mmol) in THF (10 mL) was added dropwise. The reaction was allowed to warm to room temperature and stirred for 30 minutes before being cooled to 0° C. and treated dropwise with a solution of 4-(methylthio)benzonitrile (2.91 g, 19.5 mmol) in THF (10 mL). Upon complete addition, the reaction was heated at reflux for 14 hours. After cooling, water (200 mL) was added and the mixture extracted with ethyl acetate (250 mL). The organic phase was dried over sodium sulfate, filtered and concentrated in vacuo and the resulting residue purified by silica chromatography eluting with 1:1 ethyl acetate/cyclohexane to give the title product (2.43 g) LC retention time 3.10 mins MS m/z 286 (MH⁺).

### (ii) 4-(Trifluoromethyl)-6-[4-(methylsulfonyl)phenyl]-2-pyridone

To a stirred mixture of intermediate (i) (2.43 g, 8.52 mmol) in methanol (100 mL) at 0° C. was added portionwise a suspension of Oxone™ (15.7 g, 25.6 mmol) in water (60 mL). The reaction was warmed to room temperature and stirred for 14 hours. The methanol was removed in vacuo and the resulting residue partitioned between saturated aqueous sodium bicarbonate(500 mL) and chloroform (200 mL) and separated. The aqueous layer was further extracted with chloroform (3×100 mL) and the combined organic layers were dried over sodium sulfate, filtered and concentrated to give the title compound (1.72 g) LC retention time 2.57 mins, MS m/z 318 (MH⁺).

### (iii) 2-[4-(methylsulfonyl)phenyl]-6-[(2-pyridinylmethyl)oxy]-4-(trifluoromethyl)pyridine

Diisopropylazodicarboxylate (0:93 mL, 4.7 mmol) was added dropwise to a solution of intermediate (ii) (1 g, 3.2 mmol), 2-pyridinylmethanol (0.38 mL, 3.9 mmol) and triphenylphosphine (1.249, 4.7 mmol) in chloroform (80 mL). After stirring for 14 hours, the reaction was concentrated and the residue diluted with methanol and loaded onto a methanol-conditioned 10 g Varian bond-elut SCX-2 cartridge. The cartridge was washed with methanol (2×40 mL) followed by a solution of 9:1 methanol/2N hydrochloric acid. The combined acidic fractions were concentrated and the residue triturated with methanol to give the title compound as its hydrochloride salt (348 mg) LC retention time 3.35 mins, MS m/z 409 (MH⁺); ¹H-NMR (d₆-DMSO) δ 3.28 (3H, s), 5.79 (2H, s), 7.47 (1H, s), 7.64 (1H, t, J=6 Hz), 7.85 (1H, d, J=8 Hz), 8.03 (2H, d, J=9 Hz), 8.11 (1H, s), 8.17 (1H, t, J=8 Hz), 8.38 (2H, d, J=9 Hz), 8.75 (1H, d, J=6 Hz)

### Example 3.3

### 4-methyl-N-[(1-methyl-1H-pyrazol-4-yl)methyl]-6-[4-(methylsulfonyl)phenyl]-2-pyridinamine

### (i) 4-Methyl-6-[4-(methylthio)phenyl]-2-pyridone

To a stirred solution of lithium diisopropylamide (50 mL of a 2M solution in heptane/THF/ethyl benzene, 0.1 mol) in THF (50 mL) at −78° C. and under an atmosphere of nitrogen was added dropwise a solution of 3-methyl-2-butenoic acid (5 g, 0.05 mol) in THF (50 mL). The reaction was warmed to 0° C. for 30 minutes. After cooling to −78° C., a solution of 4-(methylthio)benzonitrile (7.45 g, 0.05 mol) in THF (50 mL) was added dropwise. Upon complete addition, the reaction was warmed to room temperature and stirred for 3 hours. Water (150 mL) and ethyl acetate (100 mL) were added to the reaction mixture and the resulting precipitate filtered, washed with ethyl acetate and dried to give the title compound (4.96 g, 43%) LC retention time 2.75 mins, MS m/z 232 (MH^{+).}

### (ii) 4-Methyl-6-[4-(methylsulfonyl)phenyl]-2-pyridone

To a stirred mixture of intermediate (i) (3.7 g, 160 mmol) in methanol (150 mL) at 0° C. was added portionwise a suspension of Oxone™ (29.5 g, 48.0 mmol) in water (100 mL). The reaction was warmed to room temperature and stirred for 14 hours. The methanol was removed in vacuo and the resulting residue partitioned between saturated aqueous sodium bicarbonate(1 L) and chloroform (500 mL) and separated. The aqueous layer was further extracted with chloroform (3 ×200 mL) and the combined organic layers were dried over sodium sulfate, filtered and concentrated to give the title compound (3.20 g, 76%) LC retention time 2.20 mins, MS m/z 264 (MH⁺).

### (iii) 4-Methyl6-[4-(methylsulfonyl)phenyl]pyridine-2-trifluoromethanesulfonate

To a stirred solution of intermediate (ii) (3.20 g, 12.2 mmol) in pyridine (150 mL) at 0° C. and under an atmosphere of nitrogen was added dropwise trifluoromethanesulfonic anhydride (2.46 mL, 14.6 mmol). After stirring for 1 hr at 0° C., the pyridine was removed in vacuo and the residue partitioned between water (200 mL) and dichloromethane (200 mL). The layers were separated and the aqueous phase further extracted with dichloromethane (3×100 mL). The combined organic layers were dried over sodium sulfate, filtered and concentrated in vacuo to give the title compound (4.27 g, 89%) LC retention time 3.48 mins, MS m/z 396 (MH⁺).

### (iv) N-[(1-methyl-1H-pyrazol-4-yl)methyl]-4-methyl-6-[4-(methylsulfonyl)phenyl]pyridine-2-amine

A stirred solution of intermediate (iii) (1.25 g, 3.15 mmol) and (1-methyl-1H-pyrazol-4-yl)methylamine (0.70 g, 6.30 mmol) in NMP (10 mL) was heated at 180° C. for 14 hours, cooled, and loaded evenly onto 5 methanol-conditioned 10 g Varian bond-elut SCX-2 cartridge. The cartridges were washed with methanol (2×40 mL each) followed by a solution of 9:1 methanol/concentrated ammonium hydroxide (2×40 mL each). The ammoniacal fractions were concentrated and purified by silica chromatography eluting with a gradient of cyclohexane to ethyl acetate to give the title compound (780 mg) LC retention time 2.32 mins, MS m/z 357 (MH⁺); ¹H-NMR (CDCl₃) δ 2.23 (3H, s), 3.09 (3H, s), 3.88 (3H, s), 4.47 (2H, d, J=6 Hz), 4.68 (1H, br), 6.28 (1H, s), 6.99 (1H, s), 7.36 (1H, s), 7.50 (1H, s), 8.00 (2H, d, J=9 Hz), 8.19 (2H, d, J=9 Hz).

### EXAMPLE 4

### Biological Data

Inhibitory activity against human COX-1 and COX-2 was assessed in COS cells which had been stably transfected with cDNA for human COX-1 and human COX-2. 24 Hours prior to experiment, COS cells were transferred from the 175 cm² flasks in which they were grown, onto 24-well cell culture plates using the following procedure. The incubation medium (Dulbecco's modified eagles medium (DMEM) supplemented with heat-inactivated foetal calf serum (10% v/v), penicillin (100 IU/ml), streptomycin (100 μg/ml) and genebcin (600 μg/ml)) was removed from a flask of confluent cells (1 flask at confluency contains approximately lx107 cells). 10 mI of phosphate buffered saline (PBS) was added to the flask to wash the cells. Having discarded the PBS, cells were then rinsed in 10 ml trypsin for 20 seconds, after which the trypsin was removed and the flask placed in an incubator (37°) for 1-2 minutes unbil cells became detached from the flask. The flask was then removed from the incubator and cells resuspended in 10 ml of fresh incubation medium. The contents of the flask was transferred to a 250 ml sterile container and the volume of incubation medium subsequently made up to 100 ml. 1 ml cell suspension was pipetted into each well of 4×24-well cell culture plates. The plates were then placed in an incubator (37° C., 95% air/5% CO₂) overnight. If more than 1 flask of cells were required, the cells from the individual flasks were combined before being dispensed into the 24-well plates.

Following the overnight incubation, the incubation medium was completely removed from the 24-well cell culture plates and replaced with 250 μl fresh DMEM (37° C.). The test compounds were made up to 250× the required test concentration in DMSO and were added to the wells in a volume of 1 μl. Plates were then mixed gently by swirling and then placed in an incubator for 1 hour (37° C., 95% air/5% CO₂). Following the incubation period, 10 μl of arachidonic acid (750 μM) was added to each well to give a final arachidonic acid concentration of 30 μM. Plates were then incubated for a further 15 minutes, after which the incubation medium was removed from each well of the plates and stored at −20° C., prior to determination of prostaglandin E₂ (PGE2) levels using enzyme immunoassay. The inhibitory potency of the test compound was expressed as an IC₅₀ value, which is defined as the concentration of the compound required to inhibit the PGE2 release from the cells by 50%. The selectivity ratio of inhibition of COX-1 versus COX-2 was calculated by comparing respective IC₅₀ values.

The following IC₅₀ values for inhibition of COX-2 and COX-1 were obtained for compounds of the invention:

| Compound No. | COX-2: IC₅₀(nM) | COX-1: IC₅₀(nM) |
|---|---|---|
| 1.1 | <1 | 81,300 |
| 1.2 | 23 | 9,675 |
| 1.3 | 4 | 2,923 |
| 1.5 | 6 | 61,380 |
| 2.1(v) | 35 | >100,000 |
| 2.2(ii) | <10 | 3,880 |
| 2.3(ii) | 3 | >100,000 |
| 2.4(iii) | 370 | >100,000 |
| 2.5 | 21 | >100,000 |
| 2.6(iv) | 0.44 | 3828 |
| 2.7(viii) | 16 | >55,200 |

### EXAMPLE 5

### Microsomal Assay

Inhibitory activity against microsomal h-COX2 was assessed against a microsomal preparation from baculovirus infected SF9 cells. An aliquot of microsomal preparation was thawed slowly on ice and a 1/40,000 dilution prepared from it into the assay buffer (sterile water, degassed with argon containing 100 mM HEPES (pH 7.4), 10 mM EDTA (pH7.4), 1 mM phenol, 1 mM reduced glutathione, 20 mg/ml gelatin and 0.001 mM Hematin). Once diluted the enzyme solution was then sonicated for 5 seconds (Branson sonicator, setting 4, 1 cm tip) to ensure a homogeneous suspension. 155 μl enzyme solution was then added to each well of a 96-well microtitre plate containing either 5t1 test compound (40× required test concentration) or 5 μl DMSO for controls. Plates were then mixed and incubated at room temperature for 1 hour. Following the incubation period, 40 μl of 0.5 μM arachidonic acid was added to each well to give a final concentration of 0.1 μM. Plates were then mixed and incubated for exactly 10 minutes (room temperature) prior to addition of 25 μl 1M HCl (hydrochloric acid) to each well to stop the reaction. 25 μl of 1M NaOH (sodium hydroxide) was then added to each well to neutralise the solution prior to determination of PGE₂ levels by enzyme immunoassay (EIA).

The following IC₅₀ values for inhibition of COX-2 and COX-1 were obtained from the microsomal assay for compounds of the invention:

| Example No. | COX-2: IC₅₀(nM) | COX-1: IC₅₀(nM) |
|---|---|---|
| 1.6 | <10 | 3,752 |
| 1.7 | <10 | 79,889 |
| 1.8 | <10 | 1,860 |
| 1.9 | 22 | 69,000 |
| 1.10 | 22 | >30000 |

Examples 3.1, 3.2, 3.3 had IC50 values for inhibition of COX-2 of 0.5 μM or less and at least a 100-fold selectivity for COX-2 over COX-1,based on comparison of the respective IC₅₀ values.

### EXAMPLE 6

### Patient Study

In the following, the invention will be discussed in more detail with reference to a patient study. Other embodiments within the scope of the claims herein will be apparent to one skilled in the art from consideration of the specification or practice of the invention as disclosed herein. The results of the patient study are graphically represented in the attached figures, which will be discussed in more detail in the following.

The study may be performed as a multicenter, double-blind, placebo controlled randomised, parallel group determination of efficacy of compound 1-3 in combination with risperidone vs risperidone with placebo.

The patients may receive 2-6 mg/day of risperidone (Risperdal (E)), and, depending on which group they belonged, a therapeutically effective amount 2-butoxy4-[4-(methylsulfonyl)pheny]-6-(trifluoromethyl)pyrimidine once daily or placebo over 12 weeks after a brief wash-out period of earlier antipsychotic medication.

During the wash-out period, a benzodiazepine preparation (mostly lorazepam) may be prescribed, if necessary. Patients with agitation, anxiety, or sleeping problems may be also medicated with lorazepam during the study.

Efficacy and tolerability of the compound 2-butoxy4-[4-(methylsulfonyl)phenyl]-6-(trifluoromethyl)pyrimidine/risperidone vs placebo/risperidone will be assessed using the following endpoints—positive and negative syndrome scale (PANSS), Clinical Global Impression score (CGI), AIMS, Simpson and Angus, Bames Akathisia, Calgary Depression Scale and cognition endpoints.

The use of biperiden may be monitored as a possible indicator for side effects of the antipsychotic medication.

In order to exclude the chance that possible differences in the therapeutic effectiveness between the two groups might be due to noncompliance during the risperidone therapy or to differences in risperidone metabolism, the plasma levels of risperidone or 9-OH-risperidone may be monitored during the study.

The statistics may be performed according to the criterion of “last observation carried forward” (LOCF), i. e., the last PANSS scores of the patients who dropped out before the end of the study were carried forward to all subsequent observation days. For the comparison of the main efficacy parameter, the mean change in the PANSS between the two treatment groups, t-tests for independent samples may be employed. With reference to the underlying hypothesis of a better outcome of the compound 1-3 risperidone group, a significance of p <0.05 may be calculated in the one-tailed t-test and used as the basis for the estimation of the sample size (statistical power) and for the comparison of the groups. For all other comparisons, two-tailed t-tests may be used.

The improved effectiveness of the combination therapy with 2-butoxy4-[4-(methylsulfonyl)phenyl]-6-(trifluoromethyl)pyrimidine /risperidone in comparison to risperidone monotherapy may be clearly shown by the significantly lower PANSS global scores after the 2^{nd} to 12 weeks of treatment.

Therefore, it could be excluded that the observed differences in the therapeutic effectiveness between the two groups may be due to incompatibility during the risperidone therapy or differences in risperidone metabolism.

The combination of 2-butoxy4-[4-(methylsulfonyl)phenyl]-6-(trifluoromethyl)pyrimidine/risperidone and risperidone according to the present invention may show improved results compared to the monopreparation risperidone with regard to effectiveness in the treatment of schizophrenia.

The combination of COX-2 inhibitor as defined above and risperidone according to the present invention thus may show improved results compared to the monopreparation risperidone with regard to effectiveness in the treatment of schizophrenia.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

What is claimed is:

1. A method for the treatment of a schizophrenic disorder in a mammal in need thereof said method comprising administering to said mammal an effective amount of of a compound of formula (I) 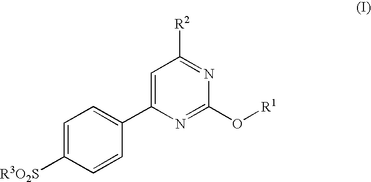
or a pharmaceutically acceptable salt or solvate thereof, in which:
R¹ is selected from the group consisting of H, C₁₋₆-alkyl, C₁₋₂alkyl substituted by one to five fluorine atoms;, C₃₋₆alkenyl, C₃₋₆alkynyl, C₃₋₁₀cycloalkylC₀₋₆alkyl, C₄₋₁₂bridged cycloalkyl, A(CR⁴R⁵)ₙ and B(CR⁴R⁵)ₙ;
R² is C₁₋₂alkyl substituted by one to five fluorine atoms;
R³ is selected from the group consisting of C₁₋₆alkyl, NH₂ and R⁷CONH;
R⁴ and R⁵ are independently selected from H or C₁₋₆alkyl;
A is selected from the group consisting of unsubstituted 5- or 6-membered heteroaryl, unsubstituted 6-membered aryl, 5- or 6-membered heteroaryl substituted by one or more R⁶ and 6-membered aryl substituted by one or more R⁶;
R⁶ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B is a ring selected from the group consisting of 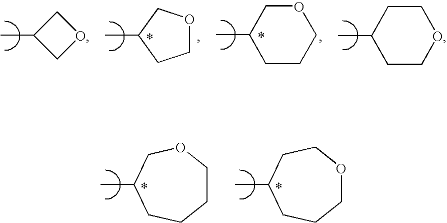
where 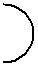
defines the point of attachment of the ring;
R⁷ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOC₁₋₆alkyl, C₁₋₆alkylOCO, H₂NC₁₋₆alkyl, C₁₋₆alkylOCONHC₁₋₆alkyl and C₁₋₆alkylCONHC₁₋₆alkyl; and
n is 0 to 4.

2. A method for the treatment of a schizophrenic disorder in a mammal in need thereof, said method comprising administering to said mammal an effective amount of a compound of formula (II) 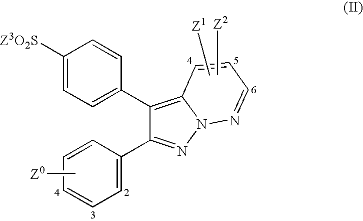
or a pharmaceutically acceptable salt or solvate thereof in which:
Z⁰ is selected from the group consisting of halogen, C₁₋₆alkyl, C₁₋₆alkoxy, C₁₋₆alkoxy substituted by one or more fluorine atoms, and O(CH₂)ₙNZ⁴Z⁵;
Z¹ and Z² are each the same or different and are independently selected from the group consisting of H, C₁₋₆alkyl, C₁₋₆alkyl substituted by one or more fluorine atoms, C₁₋₆alkoxy, C₁₋₆hydroxyalkyl, SC₁₋₆alkyl, C(O)H, C(O)C₁₋₆alkyl, C₁₋₆alkylsulphonyl, C₁₋₆alkoxy substituted by one or more fluorine atoms, O(CH₂)ₙCO₂C₁₋₆alkyl, O(CH₂)ₙSC₁₋₆alkyl, (CH₂)ₙNZ⁴Z⁵, (CH₂)ₙSC₁₋₆alkyl and C(O)NZ⁴Z⁵; with the proviso that when Z⁰ is at the 4-position and is halogen, then at least one of Z¹ and Z² is C₁₋₆alkylsulphonyl, C₁₋₆alkoxy substituted by one or more fluorine atoms, O(CH₂)ₙCO₂C₁₋₆alkyl, O(CH₂)ₙSC₁₋₆alkyl, (CH₂)ₙNZ⁴Z⁵, (CH₂)ₙSC₁₋₆alkyl or C(O)NZ⁴Z⁵;
Z³ is C₁₋₆alkyl or NH₂;
Z⁴ and Z⁵ are each the same or different and are independently selected from the group consisting of H, or C₁₋₆alkyl or, Z⁴ and Z⁵ together with the nitrogen atom to which they are bound, form a 4-8 membered saturated heterocyclic ring having 1 or 2 heteroatoms selected from N, O and S; and
n is 1-4.

3. A method for the treatment of a schizophrenic disorder in a mammal in need thereof, said method comprising administering to said mammal an effective amount of a compound of formula (III) 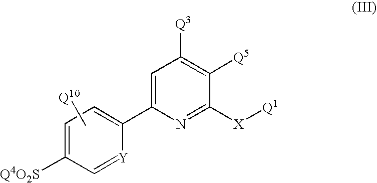
or a pharmaceutically acceptable salt or solvate thereof in which:
X is selected from the group consisting of oxygen or NQ²;
Y is selected from the group consisting of CH or nitrogen;
Q¹ is selected from the group consisting of H, C₁₋₆alkyl, C₁₋₂alkyl substituted by one to five fluorine atoms, C₁₋₃alkylOC₁₋₃alkyl, C₃₋₆alkenyl, C₃₋₆alkynyl, C₃₋₁₀-cycloalkylC₀₋₆alkyl, C₄₋₇cycloalkyl substituted by C₁₋₃alkyl or C₁₋₃alkoxy, C₄₋₁₂bridged cycloalkyl, A(CR⁶R⁷)ₙ and B(CR⁶R⁷)ₙ;
Q² is selected from the group consisting of H and C₁₋₆alkyl; or
Q¹ and Q² together with the nitrogen atom to which they are bound form a 4-8 membered saturated heterocyclic ring or a 5-membered heteroaryl ring heteroaryl ring is unsubstituted or substituted by one R⁸;
Q³ is selected from the group consisting of C₁₋₅alkyl and C₁₋₂alkyl substituted by one to five fluorine atoms;
Q⁴ is selected from the group consisting of C₁₋₆alkyl, NH₂ and R⁹CONH;
Q⁵ is selected from the group consisting of hydrogen, C₁₋₃alkyl, C₁₋₂alkyl substituted by one to five fluorine atoms, C₁₋₃alkylO₂C, halogen, cyano, (C₁₋₃alkyl)₂NCO, C₁₋₃alkylS and C₁₋₃akylO₂S;
Q⁶ and Q⁷ are independently H or C₁₋₆alkyl;
A is selected from the group consisting of unsubstituted 5- or 6-membered heteroaryl unsubstituted 6-membered aryl, 5- or 6-membered heteroaryl substituted by one or more R⁸; and 6-membered aryl substituted by one or more R⁸;
Q⁸ is selected from the group consisting of halogen, C₁₋₆alkyl, C₁₋₆alkyl substituted by one more fluorine atoms, C₁₋₆alkoxy, C₁₋₆alkoxy substituted by one or more F, NH₂SO₂ and C₁₋₆alkylSO₂;
B is a ring selected from the group consisting of 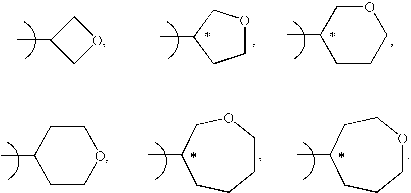
and where 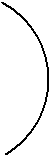
defines the point of attachment of the ring;
Q⁹ is selected from the group consisting of H, C₁₋₆alkyl, C₁₋₆alkoxy, C₁₋₆alkylOC₁₋₆alkyl, phenyl, HO₂CC₁₋₆alkyl, C₁₋₆alkylOCOCNHC₁₋₆alkyl, C₁₋₆alkylOCO, H₂NC₁₋₆alkyl, C₁₋₆alkylOCONHC₁₋₆alkyl and C₁₋₆lalkylCONHC₁₋₆alkyl;
Q¹⁰ is selected from the group consisting of H and halogen; and
n is 0 to 4.

4. The method of claim 1, further comprising administering in combination with a neuroleptic drug.

5. (canceled)

6. A method for the treatment of a schizophrenic disorder in a mammal in need thereof, said method comprising administering to said mammal an effective amount of 2-butoxy-4-[4-(methylsulfonyl)phenyl]-6-(trifluoromethyl)pyrimidine or a pharmaceutical acceptable salt or solvate thereof.

7. The method according to claim 4, characterised in that the neuroleptic is selected from clozapine, olanzapine, ziprasidone, risperidone, aripiprazole, quetiapine, quetiapine fumarate, sertindole, amisulpride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene droperidol, actophenazine, prochlorperazine, methotrimeprazine, pipotiazine, ziprasidone, hoperidone, zuclopenthixol, and mixtures thereof.

8. The method according to claim 4, wherein the neuroleptic is risperidone or aripiprazole.

9. The method of claim 1 wherein the compound is 2-butoxy-4-[4-(methylsulfonyl)phenyl]-6-(trifluoromethyl)pyrimidine or a pharmaceutical acceptable salt thereof, further comprising combination with risperidone in an amount of 0.8-3.0 mg/kg and 2-6 mg, respectively.

10. The method of claim 9, wherein risperidone is administered in an amount of 4-5 mg.

11. (canceled)

12. Kit-of-parts suitable for use in the treatment of schizophrenic disorders such as schizophrenia, delusional disorders, affective disorders, autism or tic disorders, schizophreniform disorders, in particular chronic schizophrenic psychoses and schizoaffective psychoses, temporary acute psychotic disorders, comprising, a first dosage form comprising a neuroleptic drug and a second dosage form comprising a compound of formula (I) (II) and (III) as defined in claim 1 or a pharmaceutical acceptable salt or solvate thereof, for simultaneous, separate or sequential administration.

13. Kit-of-parts according to claim 12, characterised in that the neuroleptic is selected from the group consisting of: clozapine, olanzapinea ziprasidone, risperidone, quetiapine, quetiapine fumarate, sertindole, amisulpride, haloperidol, haloperidol decanoate, haloperidol lactate, chiorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone, zuclopenthixol, and mixtures thereof.

14. Kit-of-parts according to claim 12, further comprising a compound selected from the group consisting of, celecoxib, rofecoxib, meloxicam, piroxicam, deracoxib, parecoxib, valdecoxib, etoricoxib, a chromene derivative, a chroman derivative, N-(2-cyclohexyloxynitrophenyl) methyl sulfonamide, COX189, ABT963 or JTE-522, or pharmaceutical acceptable salts or solvates thereof.

15. Kit-of-parts according to claim 12, wherein said compound is 2-butoxy-4-[4-(methylsulfonyl)phenyl]-6-(trifluoromethyl)pyrimidine or a pharmaceutical acceptable salt thereof and said neuroleptic drug is risperidone.

16. Kit-of-parts according to claim 15, wherein 2-butoxy-4-[4-(methylsulfonyl)phenyl]-6-(trifluoromethyl)pyrimidine or a pharmaceutical acceptable salt thereof and risperidone are in an amount of 0.8-3.0 mg/kg mg and 2-6 mg, respectively.

17. (canceled)

18. The method according to claim 1, wherein said mammal is human.

19. The method according to claim 18, wherein said schizophrenic disorder is selected from the group consisting of schizophrenia, delusional disorders, affective disorders, autism or tic disorders, schizophreniform disorders, in particular chronic schizophrenic psychoses and schizoaffective psychoses, temnporary acute psychotic disorders.

20. The method according to claim 19, further comprising administering a therapeutically effective amount of a a neuroleptic drug.

21. The method according to claim 20, wherein said neuroleptic drug is selected from the group consisting of: clozapine, olanzapine, ziprasidone, risperidone, quetiapine, quetiapine fumarate, sertindole, amisulpride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone, zuclopenthixol, and mixtures thereof.

22. A method for the treatment of a schizophrenic disorder in a mammal in need thereof said method comprising administering to said mammal a therapeutically effective amount of a compound, the compound is selected from the group consisting of:
2-(4fluorophenoxy)-4-[4-(methylsulfonyl)phenyl]-6](trifluoromethyl)pyrimidine;
2-(4-methoxyphenoxy)-4-[4-(methylsulfonyl)phenyl]-6-trifluoromethyl)pyrimidine;
2-butoxy-4-[4-(methylsulfonyl)phenyl]-6-(trifluoromethyl)pyrimidine;
2-[(5-chloropyridin-3-yI)oxy]-4-[4-(methylsulfony)phenyl]-6-(trifluoromethyl)pyrimidine;
2-(cyclohexyloxy)-4-[4-(methylsulfonyl)phenyl]-6-(trifluoromethyl)pyrimidine;
3-(4-methylsulfonyl-phenyl)-2-(4-methoxy-phenyl)-pyrazolo[1,5-b]pyridazine;
6-difluoromethoxy-2-(4-fluoro-phenyl)-3-(4-methylsulfonyl-phenyl)-pyrazolo[1,5-b]-pyridazine;
2-(4-ethoxy-phenyl)-3-(4-methylsulfonyl-phenyl)-pyrazolo[1,5-b]pyridazine;
2-(4-fluoro-phenyl)-6-methylsulfonyl-3-(4-methylsulfonyl-phenyl)-pyrazolo[1,5-b]pyridazine;
2-(4-difluoromethoxy-phenyl)-3-(4-methylsulfonyl-phenyl)-pyrazolo[1,5-b]pyridazine;
4-[2-(4-ethoxy-phenyl)-pyrazolo[1,5-b]pyridazin-3-yl]-benzenesulfonamide;
6-difluoromethoxy-2-(3-fluoro-phenyl)-3-(4-methylsulfonyl-phenyl)-pyrazolo[1,5-b]pyridazine;
3-(4-methanesulfonyl-phenyl)-2-(4-methoxy-phenyl)-pyrazolo[1,5-b]pyridazine;
6-difluoromethoxy-2-(4-fluoro-phenyl)-3-(4-methanesulfonyl-phenyl)-pyrazolo[1,5-b]pyridazine;
2-(4-ethoxy-phenyl)-3-(4-methanesulfonyl-phenyl)-pyrazolo[1,5-b]pyridazine;
2-(4-fluoro-phenyl)-6-methanesulfonyl-3-(4-methanesulfonyl-phenyl)-pyrazolo[1,5-b]pyridazine;
2-(4-difluoromethoxy-phenyl)-3-(4-methanesulfonyl-phenyl)-pyrazolo[1,5-b]pyridazine;
4-[2-(4-ethoxy-phenyl)-pyrazolo[1,5-b]pyridazin-3-yl]-benzenesulfonamide;
6-difluoromethoxy-2-(3-fluoro-phenyl)-3-(4-methanesulfonyl-phenyl)-pyrazolo[1,5-b]pyridazine
4-ethyl-6-[4-(methylsulfonyl)phenyl]-N-(tetrahydro-2H-pyran-4-ylmethyl)-2-pyridinamine; 4-methyl-N-[(1-methyl-1H-pyrazol-4-yl)methyl]-6-[4-(methylsulfonyl)phenyl]-2-pyridinamine;
N-[(1,5-dimethyl-1H-pyrazol-4-yl)methyl]-4-methyl-6-[4-(methylsulfonyl)phenyl]-2-pyridinamine;
N-[(1,3-dimethyl-1H-pyrazol-4-yl)methyl]-4-methyl-6-[4-(methylsulfonyl)phenyl]-2-pyridinamine;
4-(6-{[(1,3-dimethyl-1H-pyrazol-4-yl)methyl]amino}-4-ethyl-2-pyridinyl)benzenesulfonamide;
N-[(1,3-dimethyl-1H-pyrazol-4-yl)methyl]-6-[4-(methylsulfonyl)phenyl]-4-(trifluoromethyl)-2-pyridinamine;
N-[(1,5-dimethyl-1H-pyrazol-4-yl)methyl]-6-[4-(methylsulfonyl)phenyl]-4-(trifluoromethyl)-2-pyridinamine;
4-{4-methyl-6-[(tetrahydro-2H-pyran-4-ylmethyl)amino]-2-pyridinyl}benzenesulfonamide;
4-methyl-N-[(1-methyl-1H-pyrazol-3-yl)methyl]-6-[4-(methylsulfonyl)phenyl]-2-pyridinamine;
N-(cyclohexylmethyl)-6-[4-(methylsulfonyl)phenyl]-4-(trifluoromethyl)-2-pyridinamine;
N-cyclohexyl-6-[4-(methylsulfonyl)phenyl]-4-(trifluoromethyl)-2-pyridinamine;
2-[4-(methylsulfonyl)phenyl]-6-[(2-pyridinylmethyl)oxy]-4-(trifluoromethyl)pyridine;
4-methyl-N-[(3-methyl-4-isoxazolyl)methyl]-6-[4-(methylsulfonyl)phenyl]-2-pyridinamine;
6-[4-(mothylsulfonyl)phenyl]-N-(2-pyridinylmethyl)-4-(trifluoromethyl)-2-pyridinamine;
N-cycloheptyl-6-[4-(methylsulfonyl)phenyl]-4-(trifluoromethyl)-2-pyridinamine;
N-(cis-4-methylcyclohexyl)-6-[4-(methylsulfonyl)phenyl]-4-(trifluommethyl)-2-pyridinamine;
N-(1-ethylpropyl)-6-[4-(methylsulfonyl)phenyl]-4-(trifluoromethyl)-2-pyridinamine;
N-[(3-methyl-1,2,4-oxadiazol-5-yl)methyl]-6-[4-(methylsulfonyl)phenyl]-4-(trifluoromethyl)-2-pyridinamine;
N-[(5-methyl-1,2,4-oxadiazol-3-yl)methyl]-6-[4-(methylsulfonyl)phenyl]-4-(trifluoromethyl)-2-pyridinamine;
4-methyl-N-[(1-methyl-1H-pyrazol-5-yl)methyl]-6-[4-(methylsulfonyl)phenyl]-2-pyridinamine;
N-(cyclopentylmethyl)-6-[4-(methylsulfonyl)phenyl]-4-(trifluoromethyl)-2-pyridinamine;
N-[(1-ethyl-1H-1,2,4-triazol-5-yl)methyl]-4-methyl-6-[4-(methylsulfonyl)phenyl]-2-pyridinamine;
4-ethyl-6-[4-(methylsulfonyl)phenyl]-2-[(2-pyridinylmethyl)amino]-3-pyridinecarbonitrile;
4-ethyl-2-{[(5-methyl-2-pyridinyl)methyl]amino}-6-[4-(methylsulfonyl)phenyl]-3-pyridinecarbonitrile;
4-ethyl-2-{[(6-methyl-3-pyridinyl)methyl]amino}-6-[4-(methylsulfonyl)phenyl]-3-pyridinecarbonitrile;
4-ethyl-2-{[(1-methyl-1H-pyrazol-4-yl)methyl]amino}-6-[4-(methylsulfonyl)phenyl]-3-pyridinecarbonitrile;
4-ethyl-6-[4-(methylsulfonyl)phenyl]-2-{[(4-methy-1,3-thiazol-2-yl)methyl]amino}-3-pyridinecarbonitrile;
4-ethyl-6-[4-(methylsulfonyl)phenyl]-2-[(2-pyridinylmethyl)oxy]-3-pyridinecarbonitrile;
4-ethyl-N-[(1-ethyl-1H-1,2,4-triazol-5-yl)methyl]-6-[4-(methylsulfonyl)phenyl]-2-pyridinamine;
4-ethyl-2-{[(6-methyl-3-pyridinyl)methyl]oxy}-6-[4-(methylsulfonyl)phenyl]-3-pyridinecarbonitrile;
6-[4-(methylsulfonyl)phenyl]-N-[(1-methyl-1H-1,2,4-triazol-5-yl)-methyl]-4-(trifluoromethyl)-2-pyridinamine; and
pharmaceutically acceptable salts and solvates thereof

23. The method according to claim 22, wherein said mammal is human.

24. The method according to claim 23, wherein said schizophrenic disorder is selected from the group consisting of schizophrenia, delusional disorders, affective disorders, autism or tic disorders, schizophreniform disorders, in particular chronic schizophrenic psychoses and schizoaffective psychoses, temporary acute psychotic disorders.

25. The method according to claim 24, further comprising administering a therapeutically effective amount of a a neuroleptic drug.

26. The method according to claim 25, wherein said neuroleptic drug is selected from the group consisting of: clozapine, olanzapine, ziprasidone, risperidone, quetiapine, quetiapine fumarate, sertindole, amisulpride, haloperidol, haloperidol decanoate haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone, zuclopenthixol, and mixtures thereof.

27. A method for the treatment of a schizophrenic disorder in a mammal in need thereof said method comprising administering to said mammal a therapeutically effective amount of 2-butoxy-4-[4-(methylsulfonyl)phenyl]-6-(trifluoromethyl)pyrimidine or a pharmaceutical acceptable salt or solvate thereof.

28. The method according to claim 27, wherein said mammal is human.

29. The method according to claim 28, wherein said schizophrenic disorder is selected from the group consisting of schizophrenia, delusional disorders, affective disorders, autism or tic disorders, schizophreniform disorders, in particular chronic schizophrenic psychoses and schizoaffective psychoses, temporary acute psychotic disorders.

30. The method according to claim 28, further comprising administering a therapeutically effective amount of a a neuroleptic drug.

31. The method according to claim 30, wherein said neuroleptic drug is selected from the group consisting of: clozapine, olanzapine, ziprasidone, risperidone, quetiapine, quetiapine fumarate, sertindole, amisuipride, haloperidol, haloperidol decanoate, haloperidol lactate, chlorpromazine, fluphenazine, fluphenazine decanoate, fluphenazine enanthate, fluphenazine hydrochloride, thiothixene, thiothixene hydrochloride, trifluoperazine, perphenazine, amitriptyline, thioridazine, mesoridazine, molindone, molindone hydrochloride, loxapine, loxapine hydrochloride, loxapine succinate, pimozide, flupenthixol, promazine, triflupromazine, chlorprothixene, droperidol, actophenazine, prochlorperazine, methotrimeprazine, pipotiazine, ziprasidone, hoperidone, zuclopenthixol, and mixtures thereof.

32. The method according to claim 31, wherein said compound is 2-butoxy-4-[4-(methylsulfonyl)phenyl]-6-(trifluoromethyl)pyrimidine or a pharmaceutical acceptable salt thereof and said neuroleptic drug is risperidone.

33. The method according to claim 32, wherein said compound and said neuroleptic drug are present in an amount of 0.8-3.0 mg/kg mg and 2-6 mg respectively.
